(19) United States
(12) Patent Application Publication
(10) Pub. No.: US 2017/0369892 A1
(43) Pub. Date: Dec. 28, 2017

(54) HORMONE DEGRADABLE CRISPR-BASED TRANSCRIPTION FACTORS

(71) Applicant: University of Washington, Seattle, WA (US)

(72) Inventors: Eric Klavins, Seattle, WA (US); Arjun Khakhar, Seattle, WA (US); Jennifer L. Nemhauser, Seattle, WA (US)

(73) Assignee: University of Washington, Seattle, WA (US)

(21) Appl. No.: 15/630,817

(22) Filed: Jun. 22, 2017

Related U.S. Application Data
(60) Provisional application No. 62/353,403, filed on Jun. 22, 2016.

(51) Int. Cl. C12N 15/82; C12N 15/01; C12N 15/10; C12N 15/63; C12N 9/22; C12N 15/00

(52) U.S. Cl. CPC C12N 15/82; C12N 15/8213; C12N 15/63; C12N 9/22; C12N 15/01; C12N 15/102; C12N 15/00

(57) ABSTRACT

Synthetic signal transduction systems are provided. The synthetic signal transduction system may be a hormone degradable CRISPR-based transcription factor including a nuclease null Cas9 protein, a nuclear localization signal, a phytohormone degron, and a transcriptional regulation domain. Methods of generating non-naturally occurring plants are also provided. The methods may include expressing a synthetic signal transduction system in a plant. Non-naturally occurring plants formed by the methods are also provided.

### CROSS-REFERENCE TO RELATED APPLICATIONS

This application claims the benefit of and priority to U.S. Provisional Application No. 62/353,403, filed Jun. 22, 2016, which is hereby incorporated by reference in its entirety.

### STATEMENT REGARDING FEDERALLY SPONSORED RESEARCH

This invention was made with government support under Grant No. 5R01GM107084-03, awarded by the National Institutes of Health and Grant No. 1411949, awarded by the National Science Foundation. The government has certain rights in the invention.

### TECHNICAL FIELD

The present disclosure relates generally to synthetic signal transduction systems including hormone degradable CRISPR-based transcription factors. In particular, the synthetic signal transduction systems may include a nuclease null Cas9 protein, a nuclear localization signal, a phytohormone degron, and a transcriptional regulation domain. The present disclosure also relates to non-naturally occurring plants and methods of generating non-naturally occurring plants. In particular, the non-naturally occurring plants may include the synthetic signal transduction system.

### BACKGROUND

Multicellular systems in nature are capable of incredible feats of distributed computation and self-organization. Examples range from division of labor in filamentous algae (see Wilcox, M., et al. (1973) J. Cell Sci. 12, 707-723), to the exquisite sensitivity of the adaptive immune system (see Medzhitov, R. (2007) Nature 449, 819-826), to morphogenesis and development of tissues and organs. Computer scientists have shown that cells are, in principle, capable of computing a wide variety of functions (see Regot, S., et al. (2011) Nature 469, 207-11), generating complex morphologies (see Abelson, H., et al. (2000) Commun. ACM 43, 74-82), and of making decisions (see Barcena Menendez, D., et al. (2014) Curr. Opin. Biotechnol. 31C, 101-107 and Jang, S. S., et al. (2012) ACS Synth. Biol. 1, 365-374). Experimentally, synthetic multicellular systems have been built to regulate populations (see You, L., et al. (2004) Nature 428, 868-871), synchronize oscillations (see Danino, T., et al. (2010) Nature 463, 326-30), form patterns (see Chen, M.-T., et al. (2005) Nat. Biotechnol. 23, 1551-5; Sohka, T., et al. (2009) Proc. Natl. Acad. Sci. U.S.A 106, 10135-10140; and Basu, S., et al. (2005) Nature 434, 1130-4), implement logic functions through distributed computation (see Regot, S., et al. (2011) Nature 469, 207-11), and cooperate to solve problems (see Shou, W., et al. (2007) Proc. Natl. Acad. Sci. U.S.A 104, 1877-82. However, a scalable suite of cell-cell communication modules has yet to emerge. In particular, in Saccharomyces cerevisiae, strategies that use components of native signal transduction pathways can lead to crosstalk and undesirable phenotypes such as growth arrest (see Chen, M.-T., et al. (2005) Nat. Biotechnol. 23, 1551-5; Youk, H., et al. (2014) Science 343, 1242782; and Zhang, N.-N., et al. (2006) Mol. Biol. Cell 17, 3409-3422). Such systems are not obviously portable to other eukaryotes, are difficult to reprogram, and require significant changes to the host cell to function correctly (see You, L., et al. (2004) Nature 428, 868-871).

Additionally, food security is a crucial component of the economy and human health in both the developed and developing world. Based on a report from the World Bank (see “Implementing agriculture for development: World Bank Group agriculture action plan (2013-2015)”), decreases in agricultural yield lead to economic damage and higher food prices, which in turn decrease access to food among poorer segments of society leading to malnutrition.

Insects represent a significant threat to food security, globally causing, on average, a 15% loss of yield across all crops (see Maxmen, A. “Crop pests: under attack” Nature 501.7468 (2013): S15-S17), with some crops such as cotton experiencing losses of up to 80% (see Oerke, E C. “Crop losses to pests,” The Journal of Agricultural Science (2006)). This translates into huge economic losses, with the United States losing $800 million from insect damage to the 2013 maize crop alone (see Maxmen, A. “Crop pests: under attack” Nature 501.7468 (2013): S15-S17), and Brazil, which lost approximately $18 billion to crop damage by insects in 2014 (see Oliveira, C M, et al. Crop Protection 56, 50-54 (2014)). Insect related crop losses are also a serious threat to food security in a world where a 40% increase in crop yields is needed by 2050 to match the current rate of population growth. In the constant battle against agricultural pests (see Oerke, E C. “Crop losses to pests” The Journal of Agricultural Science 144.01 (2006): 31-43), one strategy that has had major success in preventing these losses is the constitutive expression of insecticidal toxins such as Bacillus thuringiensis endotoxin (BT) (see Qaim, M. et al. Science 299, 900-902 (2003)) (see FIG. 10, panel A). This is especially true in developing countries (see Qaim, M. et al. Science 299, 900-902 (2003)).

Some insects are immune to BT, such as phloem feeders like aphids. An approach to deal with them, which has not been tried in the field but has shown great promise in lab trials, is the production of insect repellent volatiles in plants (see Beale, M., et al. Proceedings of the National Academy of Sciences 103, 10509-10513 (2006)) (see FIG. 10, panel B). However, one major limitation of the current methodologies is that they all involve constitutive expression of the defense mechanisms. This constant protein or small molecule production in every cell of the plant has been shown to have detrimental effects, including yield losses of up to 25%, due to the increased metabolic burden from the unnecessary diversion of resources (see Purrington, C., et al. Genetics 145, 807-14 (1997); Brown, J. Current Opinion in Plant Biology 5, (2002); Tian, D., et al., Nature 423, 74-77 (2003); and Halfhill, M. D., et al., Molecular Ecology 14, 3177-3189 (2005).

This strategy also comes with the cost of allowing faster development of resistance among pests (see Gassmann, A. J., et al. Proc. Natl. Acad. Sci. U.S.A 111, 5141-6 (2014)) due to long term exposure of insects to the insecticidal proteins as well as environmental contamination with low concentrations of the toxin through pollen dispersal by engineered plants (see Chilcutt, C., et al. Proc. Natl. Acad. Sci. U.S.A 101, 7526-7529 (2004)). Williams, et al. have demonstrated how the expression of BT in plants could be made chemically inducible (see Nature Biotechnology 10, 540-543 (1992)), however, this approach used a toxic inducer, β-estradiol. Additionally, it only slightly mitigates the problem as it is not feasible to selectively induce plants being attacked by insects on an agricultural scale. Plants, however, naturally have the ability to not only detect the presence of insect herbivore-based damage but also to discriminate between different insect herbivores (see Vos, D. M. et al. “Signal signature and transcriptome changes of Arabidopsis during pathogen and insect attack,” MPMI Vol. 18, No. 9, 2005, pp. 923-937). This sensing is associated with fluxes of specific phytohormones.

### BRIEF DESCRIPTION OF THE DRAWINGS

The embodiments disclosed herein will become more fully apparent from the following description and appended claims, taken in conjunction with the accompanying drawings.

FIG. 1A depicts an auxin degradable CRISPR-based transcription factor (ADCTF) design and the molecular mechanism behind its function. An ADCTF can include a nuclease null Cas9 protein (dCas9) fused to a nuclear localization signal (NLS), an activation domain, and an auxin sensitive degron. In the presence of auxin, the degron can recruit an Auxin Sensing F-box (AFB) protein to form an SCF complex (an E3 ubiquitin ligase). The subsequent ubiquitination and degradation of the ADCTF can deregulate the gene targeted by the ADCTF.

FIG. 1B is a graph depicting the results of time-lapse cytometry of ADCTF cells with a GFP-producing guide RNA (gRNA) target following the addition of auxin or no treatment as well as with and without a gRNA. The dashed-line ribbon indicates the 95% confidence interval. Following treatment with auxin, the GFP level of the strain expressing gRNA dropped to basal levels (equivalent to a strain with no gRNA).

FIG. 1C is a schematic representation of the three fusion proteins tested for the effect of degron position on ADCTF properties.

FIG. 1D depicts sensitive range characterization of the three degron position variants of FIG. 1C at steady state. Horizontal bars indicate the range of auxin concentrations between which mean steady-state fluorescence (measured via cytometry) drops from 90% of maximum to 10%. A larger sensitive range correlated with higher maximum fold changes upon induction (see FIGS. 7A and 7B).

FIG. 2A illustrates that the ADCTF (auxin receiver) strain library was generated from all pairwise combinations of three auxin sensing F-box protein variants (AFB2, TIR1, and tir1-D/M) and three auxin degron variants (from IAA14, IAA15, and IAA17).

FIG. 2B is a graph depicting receiver strain library degradation kinetics measured via time-lapse cytometry. The kinetics of ADCTF responses to auxin were characterized by the time at which fluorescence dropped to fifty percent of maximum (a smaller time implies a faster response). The ADCTF library displays a wide range of degradation kinetics that were modulated by both the choice of F-box protein and the auxin degron.

FIG. 2C is a graph depicting auxin sensitivity ranges for the ADCTF library. The horizontal bars represent the auxin sensitivity range at steady state as defined in FIGS. 1A-1D.

FIG. 3A illustrates an auxin sender strain design. The iaaH enzyme of Agrobacterium tumefaciens catalyzes the conversion of indole-3-acetamide (IAM) into auxin, inducing the degradation of proteins fused to an auxin degron. The iaaH enzyme (sender cells) was integrated into an auxin reporting strain (the EYFP-IAA17|AFB2 strain from Havens, et al. (2012) Plant Physiol. 160, 135-42) to test for internal auxin production.

FIG. 3B is a graph depicting kinetic auxin response to IAM addition in sender strains. Following the addition of IAM, the fluorescence of sender cells decreased to basal levels. The time to half-maximal (t_{1/2}) fluorescence was used to measure the rate of reporter degradation.

FIG. 3C is a graph depicting auxin-induced degradation rate in response to varying doses of either IAM or auxin. Sender cells were treated with either auxin or IAM and read at regular intervals producing time courses as in FIG. 3B. Nonlinear fitting was used to generate t_{1/2} values. For a given molarity, treatment with IAM produces an auxin-induced degradation similar to, but weaker than, direct treatment with auxin.

FIG. 3D is a graph depicting steady state fluorescence in response to varying doses of either IAM or auxin taken from the same dataset as in FIG. 3C. As the concentration of IAM was increased, a lower steady state fluorescence was produced.

FIG. 4A illustrates sender-sensor multicellular auxin signaling strains. Sender cells are identical to those in FIGS. 3A-3D and therefore produce auxin upon the addition of exogenous IAM and sense auxin production via an EYFP-IAA17 reporter. Sensor cells express an EYFP-IAA17 and TIR1 and are distinguished experimentally through the expression of mCherry. In coculture, IAM diffuses into sender cells where it is converted into diffusible auxin that then degrades EYFP-degron proteins in either the sender or sensor cell types.

FIG. 4B is a graph depicting auxin-induced degradation of EYFP-IAA17 in sensor cells cocultured with sender cells in 300 μM IAM. Data for sensor cells can be separated from sender cells via their mCherry signal. The line represents a LOESS fit and the dashed-line ribbon represents a 95% confidence interval of the fit.

FIG. 4C, left graph, is a graph depicting sender cell fraction dose response. Each fraction had the same volume, so a larger fraction indicates a larger concentration of sender cells in coculture. As the sender cell population increases, the degradation rate decreases. The graph on the right depicts steady state fluorescence in response to varying doses of either IAM or auxin taken from the same dataset as in the graph on the left. As the concentration of sender cells was increased, a lower steady state fluorescence was produced, flatting out at around a 50:50 split.

FIG. 5A illustrates coculture of sender and receiver strains. Sender cells convert IAM into auxin that then diffuses out of sender cells and into receiver cells where it causes the degradation of ADCTFs, producing a drop in fluorescence.

FIG. 5B is a graph depicting time course data for two replicates (shown in white and black dots) of a coculture of equal concentrations of sender and receiver cells is plotted on the left. The line represents a LOESS fit and the dashed-line ribbon represents a 95% confidence interval of the fit. On the right, histograms display distinct populations of sender (dashed line, left) and receiver (solid line, right) cells. In the presence of sender cells treated with IAM, receiver cells dropped in fluorescence over time. As in FIGS. 3A-3D and 4A-4C, sender cells also express an EYFP-IAA and AFB auxin reporter and therefore also show a decrease in fluorescence. Without IAM, receiver cells did not show a significant decrease in fluorescence.

FIG. 5C is a graph depicting degradation rates (measured as t_{1/2}) in receiver strains in response to sender cell concentration. As the fraction of sender cells increased, there is a more dramatic auxin effect in receiver cells that saturates at approximately even fractions of send to receive.

FIG. 5D is a graph describing change in fluorescence in receiver strains in response to sender cell concentration. As the fraction of sender cells increased, there is a more dramatic auxin effect in receiver cells that saturates at approximately even fractions of send to receive.

FIG. 6A is a graph depicting raw data of a time course where receiver cells with ADC17 and AFB2 were treated with auxin at time zero and then fluorescence was observed until the sample reached steady state, after which they were washed to remove auxin and recovery was observed over a similar period as induction. The lines are LOESS fits to the data and the dashed-line ribbon represents a 95% confidence interval of the fit.

FIG. 6B is a graph depicting raw data of two time course replicates of receiver strains with an ADC transcription factor (TF) with degron 17 and either AFB2 or TIR1-DM F-boxes. Both these strains have a minimal pGAL promoter driving GFP production and a gRNA that targets this promoter. Cultures treated with 30 μM auxin show a comparable release of regulation to when pCYC1 was targeted with the ADC TF. The lines are LOESS fits to the data and the dashed-line ribbon represents a 95% confidence interval of the fit.

FIG. 7A is a graph depicting raw data of two time course replicates of the three positional variants of the ADC TFs for cultures that were treated with 10 μM auxin, as well as parallel untreated controls. The auxin treated cultures have a consistent drop in fluorescence, with position one having the largest drop at steady state. Position three has no noticeable drop at this auxin concentration. The lines are LOESS fits to the data and the dashed-line ribbon represents a 95% confidence interval of the fit.

FIG. 7B is a graph depicting raw data of two dose response replicates of the three positional variants of the ADC TFs five hours after induction with auxin. Position 1 has the highest sensitivity to auxin, and consequently saturates first, followed by position 2 and then position 3. The lines are LOESS fits to the data and the dashed-line ribbon represents a 95% confidence interval of the fit.

FIG. 8A is a graph depicting raw data of two dose response replicates of all possible pairwise combinations of different degrons on ADC TFs with the three F-boxes, AFB2, TIR1, and TIR1-DM, twenty-four hours after induction with auxin. A range of auxin sensitivities are represented in the library, TIR1 being the most insensitive, and AFB2 and TIR1-DM being much more sensitive, depending on the degron being used. The lines are LOESS fits to the data and the dashed-line ribbon represents a 95% confidence interval of the fit.

FIG. 8B is a graph depicting raw data of two time course replicates of all possible pairwise combination of different degrons on ADC TFs for cultures that were treated with 30 μM auxin at time zero. A range of different degradation kinetics are observed with some reaching steady state within 400 minutes such as IAA15+TIR1-DM, whereas other are still decreasing. The lines are LOESS fits to the data and the dashed-line ribbon represents a 95% confidence interval of the fit.

FIG. 8C is a graph depicting normalized data of the two time course replicates of FIG. 8B. As stated above, a range of different degradation kinetics are observed with some reaching steady state within 400 minutes such as IAA15+TIR1-DM, whereas other are still decreasing. The lines are LOESS fits to the data and the dashed-line ribbon represents a 95% confidence interval of the fit.

FIG. 8D is a graph summarizing the raw time course data from FIG. 8C by plotting the T 0.5, time taken for the fluorescence to drop to fifty percent of its maximal drop, versus the percentage drop at steady state, measured twenty-four hours after induction. While there appears to be a large range of degradation rates achievable by using different combinations of degrons and ADC-TFs, the steady state percentage changes are all approximately equal, with a variance of 20% between the highest and the lowest, but with most clustering at approximately 75%.

FIG. 9 is a graph depicting normalized time course data for two replicates of sender receiver coculture experiments corresponding to FIGS. 5A-5C. Sender fractions are indicated by different fill patterns. Lines represent LOESS fits to the combined mean fluorescence data from both replicates. Auxin treatment and IAM treatment is represented as indicated.

FIG. 10 is a graphic explanation of pest triggered immunity. Panel A is a schematic of how current traditional constitutive expressing BT crops work. The BT is represented by the patterned hexagons and the pattern in the plants. After eating the plant which is producing the BT, the chewing insect herbivore shown in green dies. Panel B is a schematic of how crops constitutively expressing insect pheromones would work. A synthase gene, turns a secondary metabolite, shown as triangles, to aphid alarm pheromone, shown as circles and the diffuse gas around the plants. In the presence of the alarm pheromone, aphids are repelled and their natural predators are attracted. Panel C is an explanation of how the pest triggered immunity system would work for chewing pests. Chewing herbivory causes a JA flux to occur, shown as the circles. The pest triggered immunity system links this JA flux to the production of BT, the hexagons, in the tissues being attacked. When the pests eat the BT they die. Panel D is an explanation of how the pest triggered immunity system works for sucking pests. Upon sucking herbivory there is a SA flux, shown as squares. The PTI system links this SA flux to the biosynthesis of the aphid alarm pheromone which repels aphids and attracts their predators. Panel E demonstrates how the PTI circuitry works. In the presence of a phytohormone the transcription factor gets ubiquitinated and subsequently degraded releasing repression of the insect defense mechanism.

FIG. 11 depicts increasing PIN1 canalization using synthetic promoters. The graphics depict promoter constructs corresponding to the data described below them. The schematic on the far right describes what the divergence angle is. Histograms describe distributions of divergence angles for plant lines where an extra copy of PIN1 driven by promoters with a range of auxin sensitives. Each histogram is aggregate data for 5 independent T1 lines with 2 stems measured per line. The wildtype maximum, 137.5 degrees, is depicted as the solid line.

FIG. 12A is a schematic of the genetic circuit that was transformed into Arabidopsis thaliana to test the function of the hormone degradable CRISPR based transcription factor (HDCTF). It shows the HDCTF being expressed from a strong constitutive UBQ10 promoter and the guide RNA (gRNA) being expressed from a U6 promoter. These complex together and cause repression of the UBQ1 promoter, which is driving the expression of a venus-luciferase fusion reporter with a nuclear localization tag fused to it. Auxin can interact with the degron domain of the ADCTF (a version of the HDCTF) to degrade the transcription factor and relieve repression.

FIG. 12B is graph showing the normalized luciferase reporter activity over time for two plants, one of which has an ADCTF with a functional degron and the other which has a ADCTF with a stabilized degron. The orange bar indicates when the plants were exposed to either an auxin (solid line) or control (dashed line) treatment. The box plot summarizes the behavior of several plant lines with the functional or stabilized degron ADCTFs eighty minutes after treatment. A significant increase in reporter expression was observed upon auxin induction in the case of the functional degron but not the control, as expected.

FIGS. 12C and 12D are confocal microscopy images showing the root tip of a plant with the circuit described in FIG. 12A in it. Upon treatment with auxin a significant increase in reporter expression was observed.

FIG. 12E is a schematic of a ratiometric reporter line built, based on the ADCTF, to visualize endogenous auxin during development. In addition to an ADCTF regulating a nuclearly localized Venus-luciferase reporter, the lines also have a nuclearly localized ndTomato reporter being driven by a version of the UBQ1 promoter with the gRNA target site mutated.

FIG. 12F depicts a root tip (left) and an elongation zone (right) of a plant line with the ratiometric auxin reporter in it visualized using confocal microscopy. The images are false colored based on the color bar on the extreme right, and the signal visualized is the background subtracted venus signal normalized by the background subtracted ndTomato signal. The patterns of auxin distribution agree with currently existing reporters.

FIGS. 12G and 12H show the role of auxin in lateral root initiation using the ratiometric reporter system with the auxin collection in the lateral root founder cells (FIG. 12G, dashed white box) and auxin maxima in the emerging lateral root (FIG. 12H, dashed white box). The images are false colored based on the color bar on the extreme right, and the signal visualized is the background subtracted venus signal normalized by the background subtracted ndTomato signal.

FIG. 13A is a schematic of how the degron domain of an HDCTF can be swapped with degrons that respond to hormones other than auxin, such as jasmonate and gibberellin.

FIGS. 13B, 13C, and 13D are box plots showing normalized luciferase reporter activity of plant lines that were treated with either a control treatment or a phytohormone. The degron used for the HDCTF in the plant lines is shown in the top left corner of the boxplot. The expected increase in reporter expression upon treatment with the appropriate phytohormone as compared to a control treatment was observed.

FIG. 13E is a schematic of how the degron domain of a ADCTF can be swapped with degrons with different sensitivities to auxin.

FIGS. 13F, 13G, and 13H are graphs showing the reporter signal over time, time to maximum response, and percent change post treatment with auxin for plant lines with the ADCTF variants that have different auxin sensitivity degrons, as described in FIG. 13E.

FIG. 13I is a schematic of how the repression domain of a ADCTF can be swapped with different repression domains, such as MXI1.

FIGS. 13J, 13K, and 13L are graphs showing the reporter signal over time, time to maximum response, and percent change post treatment with auxin for plant lines with the ADCTF variants that has a different repression domain, as described in FIG. 13E.

FIG. 13M is a schematic illustrating that different numbers of gRNAs can be used to target a promoter.

FIGS. 13N, 13O, and 13P are graphs showing the reporter signal over time, time to maximum response, and percent change post treatment with auxin for plant lines that have different numbers of gRNAs targeting the reporter promoter, as described in FIG. 13M.

FIG. 14A is a set of schematics that show the predicted effects of lowering canalization strength. It would be expected to see fewer branches on the plant, due to a lowered ability for buds to drain auxin into the central vasculatures auxin flux (shown by the orange arrows) which would lead to less branches. On a molecular level, this could be implemented by reducing how much auxin activated the expression of PIN1.

FIG. 14B depicts box plots comparing the number of branches on a Columbia control to plant lines which have an ADCTF repressing the expression of PIN1 in an auxin dependent manner (Blue). Each line is a different ADCTF background and each dot on the plot is a different line with a PIN1 targeting gRNA. The predicted decrease in the number of branches was observed.

FIG. 14C is a set of schematics that show the predicted effects of lowering the auxin activation of LBD16 by targeting it with an ADCTF. It would be expected to see fewer lateral roots.

FIG. 14D is a box plot describing the number of lateral roots normalized by length of the primary root in plant lines with gRNAs targeting the promoter of LBD16 (Blue). A decrease in the number of lateral roots as compared to a line without the gRNAs is observed.

FIG. 15A shows a schematic of the genetic circuit that was transformed into Arabidopsis thaliana, with a jasmonate degradable CRISPR-based transcription factor (JDCTF) regulating a venus-luciferase fusion reporter. Jasmonate is a plant hormone produced when plants undergo mechanical damage, for example, under insect herbivory.

FIG. 15B is a box plot that describes the fold change post mechanical damage as compared to an undamaged control leaf for several plant lines with the JDCTF system in them.

FIG. 15C, bottom panel, is a graph showing the luciferase reporter activity of a representative plant line for a leaf that was mechanically damaged (light blue) as compared to a control leaf that was not damaged (dark blue). The top panels depict bright field images of the plant in the time course at different time points with a superimposed luciferase signal false colored according to the color bar on the far right.

### DETAILED DESCRIPTION

The present disclosure relates generally to synthetic signal transduction systems including hormone degradable CRISPR-based transcription factors (HDCTFs). The synthetic signal transduction systems or HDCTFs may include a dCas9, a nuclear localization signal, a phytohormone degron, and a transcriptional regulation domain. The present disclosure also relates to non-naturally occurring plants and methods of generating non-naturally occurring plants. A synthetic signal transduction system or HDCTF may be expressed in a plant to form the non-naturally occurring plant.

It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

The terms “bind” or “bound” are used broadly throughout this disclosure to refer to any form of attaching or coupling two or more components, entities, or objects. For example, two or more components may be bound to each other via chemical bonds, covalent bonds, ionic bonds, hydrogen bonds, electrostatic forces, Watson-Crick hybridization, etc.

Orthogonality can be crucial for rationally engineering cell-cell communication. Auxin, a plant hormone, does not have measurable effects on laboratory strains of yeast (see Havens, K. a, et al. (2012) Plant Physiol. 160, 135-42 and Pierre-Jerome, E., et al. (2014) Proc. Natl. Acad. Sci. U.S.A 111, 9407-12) when grown in standard conditions. The receiver cells provided herein use elements of the Arabidopsis thaliana auxin signaling pathway. Auxin regulates plant development via a system of transcriptional corepressors, the Aux/IAA proteins (referred to as IAAs), which are degraded in the presence of the molecule auxin. Auxin stabilizes the interaction between the degron domain of an IAA and an auxin-signaling F-box protein (AFB). The result is the degradation of the IAA via polyubiquitination (see Gray, W. M., et al. (2001) Nature 414, 271-276). The IAAs exhibit a range of degradation rates and sensitivities to auxin that are determined, in part, by the sequence of their degron domains and in part by the AFB (see Havens, K. a, et al. (2012) Plant Physiol. 160, 135-42 and Villalobos, C., et al. (2012)). The degradation dynamics of a large range of auxin degrons with multiple AFBs have been previously studied and characterized in yeast (see Havens, K. a, et al. (2012) Plant Physiol. 160, 135-42). By using this signaling modality as the basis for the communication system provided herein, use of any native yeast (or mammalian) signal transduction machinery associated with adverse phenotypes is avoided (see You, L., et al. (2004) Nature 428, 868-871). Additionally, the primary components of the pathway, AFBs and IAAs, have been shown to function in several different mammalian cells (see Nishimura, K., et al. (2009) Nat. Methods 6, 917-22), suggesting that the system provided herein may be broadly portable.

To maximize modularity, auxin responsiveness was engineered into CRISPR transcription factors (CTFs). CTFs can consist of a dCas9 fused to a transcriptional effector domain. The dCas9 can be programmed to target a locus by coexpressing a small gRNA that has complementarity to the target locus at a site that is adjacent to an “NGG” sequence, called the PAM sequence. This strategy, as demonstrated by Farzadfard, et al. (see (2013) ACS Synth. Biol. 2, 604-13) and Qi, et al. (see (2013) Cell 152, 1173-1183), can have the benefit of modularity through easily programmable specificity (dCas9 generally requires only the expression of a new gRNA for retargeting). In contrast, zinc finger or TAL DNA binding domains require the design of a new protein for each target (see Khalil, A. S., et al. (2012) Cell 150, 647-658 and Kiani, S., et al. (2014) Nat. Methods 11, 723-6). These characteristics can make CTFs a candidate for signal reception and processing, as they can be targeted to any promoter in the genome that has a suitable PAM site (see Farzadfard, F., et al. (2013) ACS Synth. Biol. 2, 604-13), can either activate or repress gene expression, and can be layered to form more complex networks (see Kiani, S., et al. (2014) Nat. Methods 11, 723-6 and Nielsen, A. A. K., et al. (2014) Mol. Syst. Biol. 10, 1-12).

In the present case, CTFs fused to the VP64 strong activator domain were targeted to a promoter upstream of GFP. In addition, these CTFs were fused to Aux/IAA degron domains and co-expressed with AFBs thereby producing auxin-degradable CRISPR transcription factors, or ADCTFs. An ADCTF is thus a modular, coupled sensor actuator, which should allow cell-to-cell communication to be rewired to arbitrary outputs.

Signal production and reception in cell-cell communication can ideally be tunable to achieve a broad range of sensitivities and other functions. To implement and tune auxin production in the sender, the bacterial iaaH gene from Agrobacterium tumefaciens was integrated into yeast under the control of a constitutive promoter (GPD). Upon the addition of indole-3-acetamide (IAM), sender cells produced a strong enough auxin signal to affect gene regulation via the ADCTFs in co-cultured receiver cells. The concentration of auxin produced can be tuned via the concentration of exogenously added IAM. For increased tunability, a library of ADCTFs was developed, each with a different degron and/or degron location, which displays a range of degradation kinetics and sensitivities to auxin. The sensitivity of the ADCTFs can be further tuned by the selection of the F-box that is coexpressed with the ADCTF. Thus, components of the ADCTFs, the auxin degron, and the transcriptional effector domain can all be swapped to obtain, respectively, a range of auxin sensitivities, and repression versus activation.

Accordingly, the combination of sender and receiver modules described herein forms the foundation of an orthogonal, modular, and tunable cell-cell communication framework for yeast. Each of these aspects of the system is demonstrated below by describing how the senders and receivers behave in isolation, and that they can be combined in co-culture to form a simple communication channel.

An engineering framework for synthetic multicellular systems can require a programmable means of cell-cell communication. Such a communication system may enable complex behaviors, such as pattern formation, division of labor in synthetic microbial communities, and improved modularity in synthetic circuits. However, it remains challenging to build synthetic cellular communication systems in eukaryotes due to a lack of molecular modules that are orthogonal to the host machinery, easy to reconfigure, and scalable. Here, a novel cell-to-cell communication system in Saccharomyces cerevisiae (yeast) based on CRISPR transcription factors and the plant hormone auxin that exhibits several of these features is provided.

Specifically, a sender strain of yeast was engineered that converts indole-3-acetamide (IAM) into auxin via the enzyme iaaH from Agrobacterium tumefaciens. To sense auxin and regulate transcription in a receiver strain, a reconfigurable library of auxin degradable CRISPR-based transcription factors (ADCTFs) was engineered. Auxin-induced degradation can be achieved through fusion of an auxin sensitive degron (from IAA co-repressors) to the CRISPR TF and co-expression with an auxin F-box protein. Mirroring the tunability of auxin perception in plants, the family of ADCTFs provided herein exhibits a broad range of auxin sensitivities. The kinetics and steady state behavior of the sender and receiver were characterized independently, and in co-cultures where both cell types were exposed to IAM. In the presence of IAM, auxin is produced by the sender cell and triggers de-activation of reporter expression in the receiver cell. The result is an orthogonal, rewireable, tunable, and scalable cell-cell communication system for yeast and other eukaryotic cells.

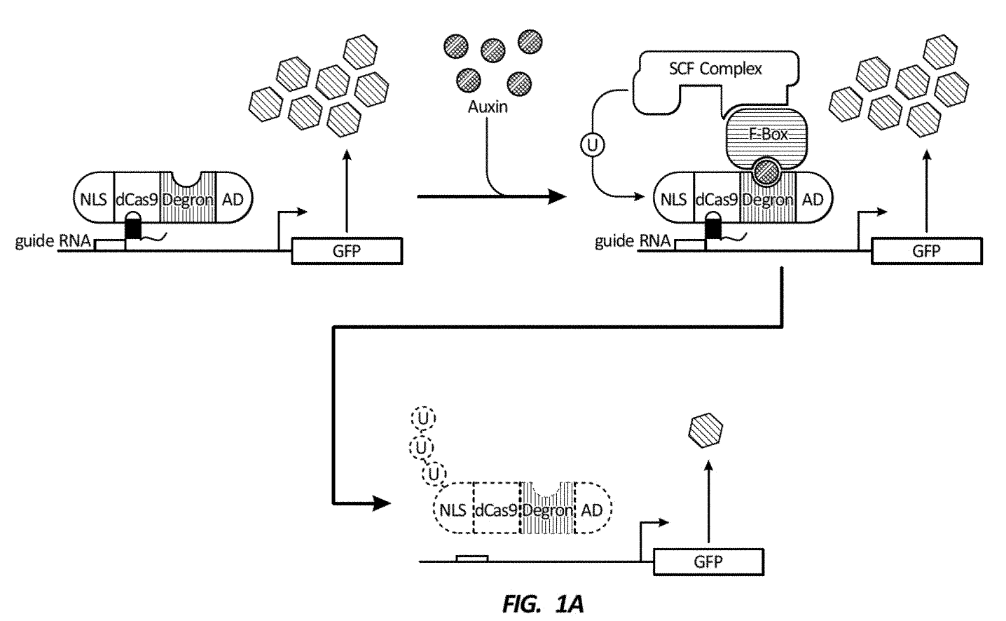

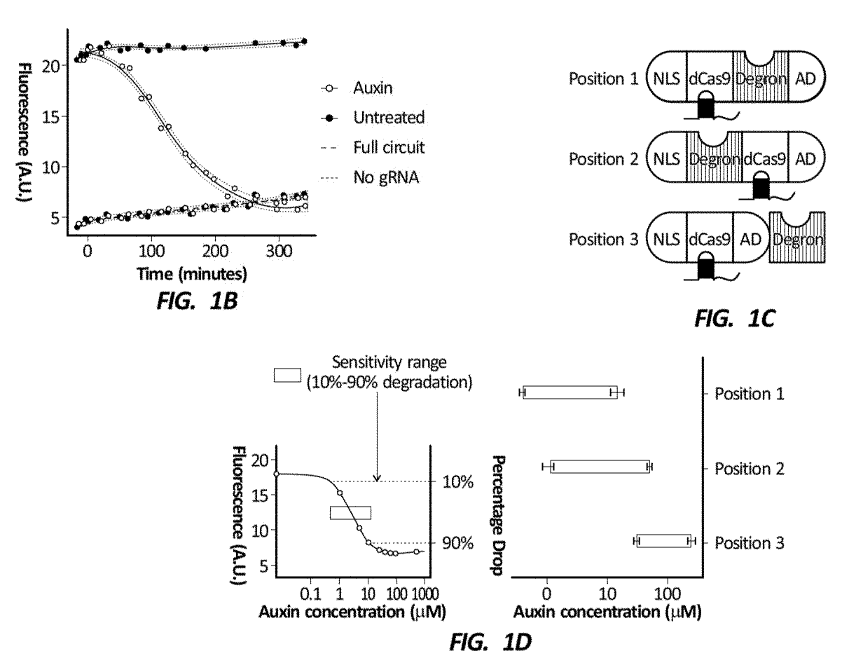

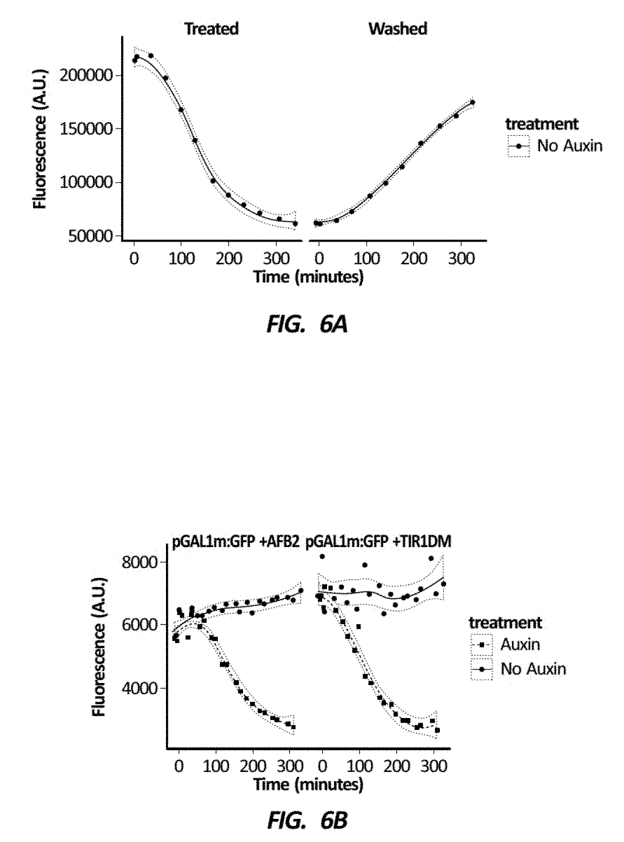

Synthetic, scalable, auxin-modulated transcription factors are provided. To link an auxin sensor to diverse transcriptional responses and targets, auxin degradable CRISPR transcription factors (ADCTFs) with three modular domains were designed (see FIG. 1A). In some embodiments, a core component of the ADCTFs can be the CRISPR-based transcription factor described by Farzadfard, et al. (“Tunable and multifunctional eukaryotic transcription factors based on CRISPR/Cas,” ACS synthetic biology 2.10 (2013): 604-613), wherein a dCas9 protein functions as a programmable DNA binding module. The dCas9 was fused to a transcriptional effector domain, in this case the transcriptional activator VP64, and to an IAA degron. In the presence of an AFB, ADCTFs should generally be ubiquitinated and degraded when exposed to auxin. The ADCTFs were tested by targeting them to activate the expression of EGFP from a minimal CYC1 promoter and observing deactivation of fluorescence upon the addition of auxin. In the absence of auxin, functional ADCTFs significantly activated the production of EGFP as compared to controls lacking a gRNA (see FIG. 1B). When a functional (coexpressed with gRNA) activator ADCTF was degraded in the presence of auxin, fluorescence dropped to levels at or below the control (no gRNA) levels. Auxin dependent regulation was independent of the promoter being regulated by the ADCTF (see FIG. 6B). The observed effect was also reversible. When auxin was removed from the system, reporter expression returned to its activated state (see FIG. 6A).

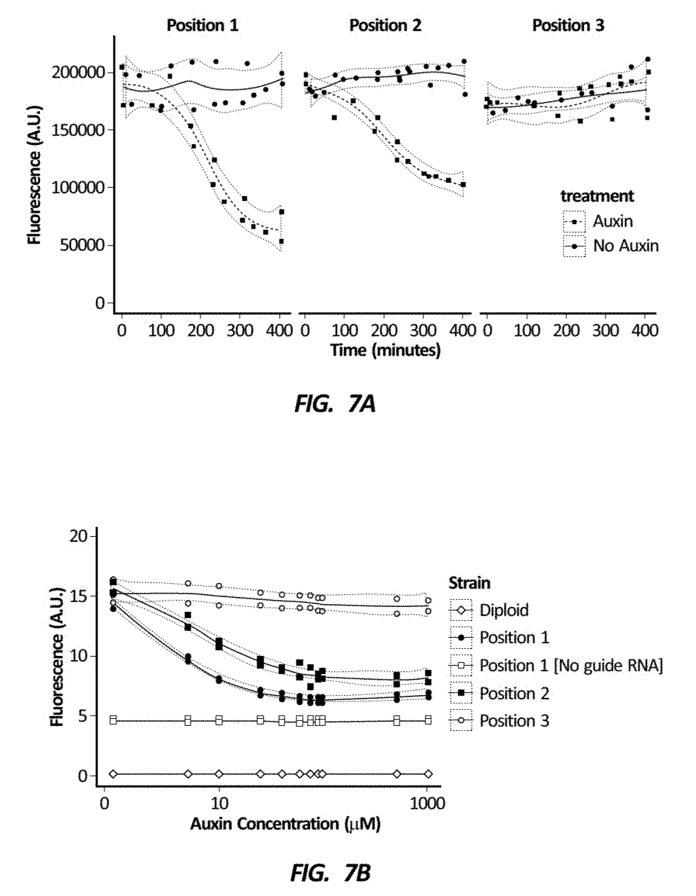

One design consideration for building the ADCTFs was the position of the degron within the fusion protein. Without being bound by any one specific theory, it was hypothesized that degron position could alter accessibility to the AFB or otherwise interfere with protein folding, thus modulating auxin sensitivity. Several possible positions for the degron relative to the other domains were explored (see FIG. 1C). In all cases, the degron was flanked by flexible linkers composed of five repeats of the amino acid sequence “GS” to limit fusion-associated misfolding. Changing the position of the degron dramatically altered the sensitivity range, defined as the range of auxin concentrations between which steady-state fluorescence drops from 90% of maximum to 10% (see FIG. 1D). Position one is sensitive to the lowest levels of auxin, but also saturates earlier than positions two and three. Placing the degron on either side of dCas9 (positions one and two) resulted in higher auxin sensitivity than position three where the degron was placed at the C-terminal end of the fusion. The percentage drop from maximal activation upon auxin induction was directly correlated to auxin sensitivity, with position one dropping to basal levels at steady state, and positions two and three having progressively smaller effects post induction (see FIGS. 7A and 7B). Altering the position of the degron coarsely tuned the upper and lower bounds of the sensitivity range of the ADCTF. However, since the position one variant was the most sensitive to auxin and had the highest fold change, degrons in all further ADCTF variants were fused at position one.

Engineered ADCTF variants can exhibit a broad range of auxin sensitivities and degradation kinetics. The Aux/IAA family of 29 transcriptional corepressors have been shown to exhibit a large range of degradation rates and sensitivities to auxin in yeast (see Havens, K. a, et al. (2012) Plant Physiol. 160, 135-42). Without being bound by any one specific theory, this range of responses to the same auxin signal may result in part from the sequence of the different IAA degron domains, and in part from the varying activities of different AFBs, each showing different affinities for specific IAAs. A library of ADCTFs was built using degrons from IAA14, IAA15, and IAA17 and coexpressing them with either of two F-boxes (AFB2 or TIR1). These degrons have been previously characterized as encompassing a range of auxin-induced degradation rates. In general, AFB2 promotes faster degradation of IAAs than TIR1. In addition, a recently characterized mutant of TIR1, tir1-D170E/M473L (also referred to herein as tir1-D/M) was included (see Yu, H., et al. (2013) Plant Physiol. 162, 295-303), which has been shown to greatly accelerate auxin-induced TIR1 degradation.

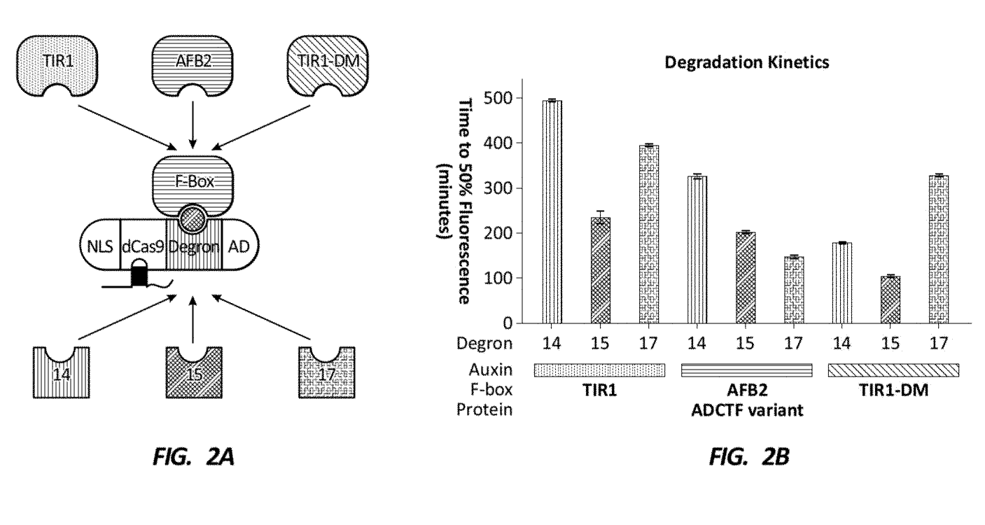

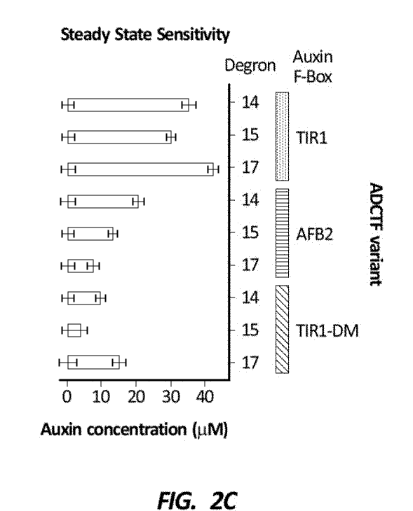

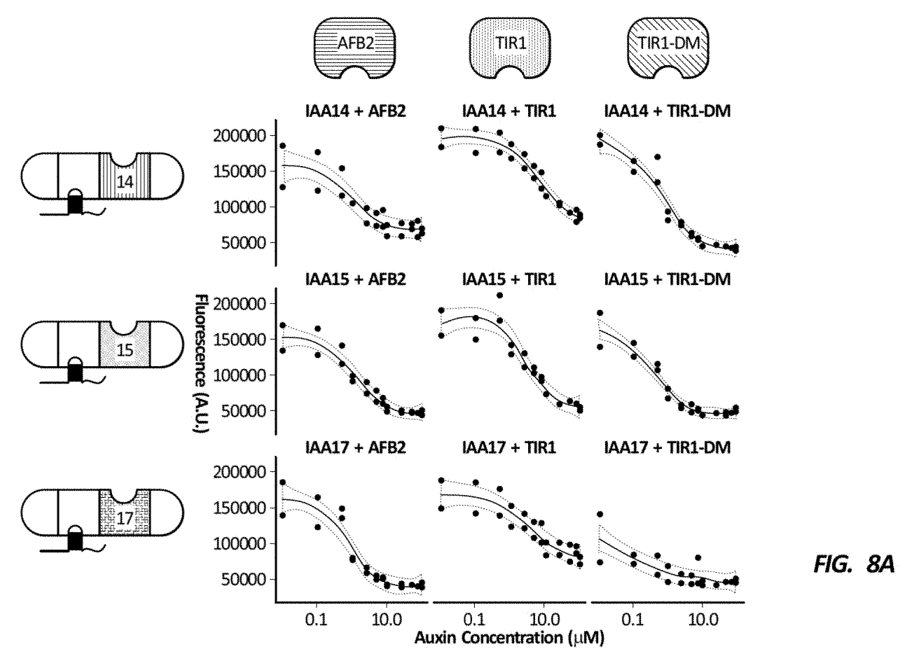

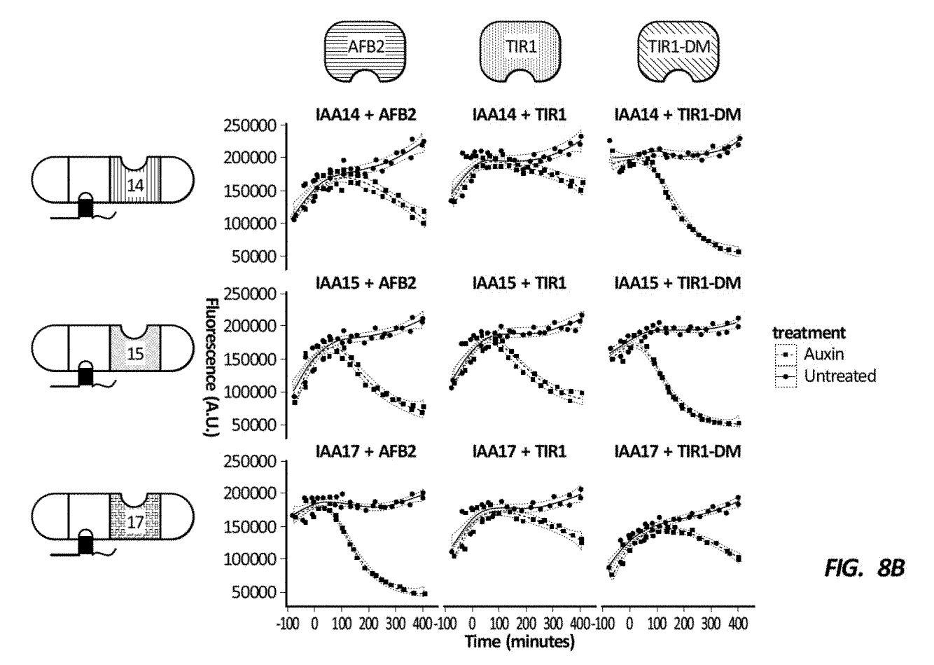

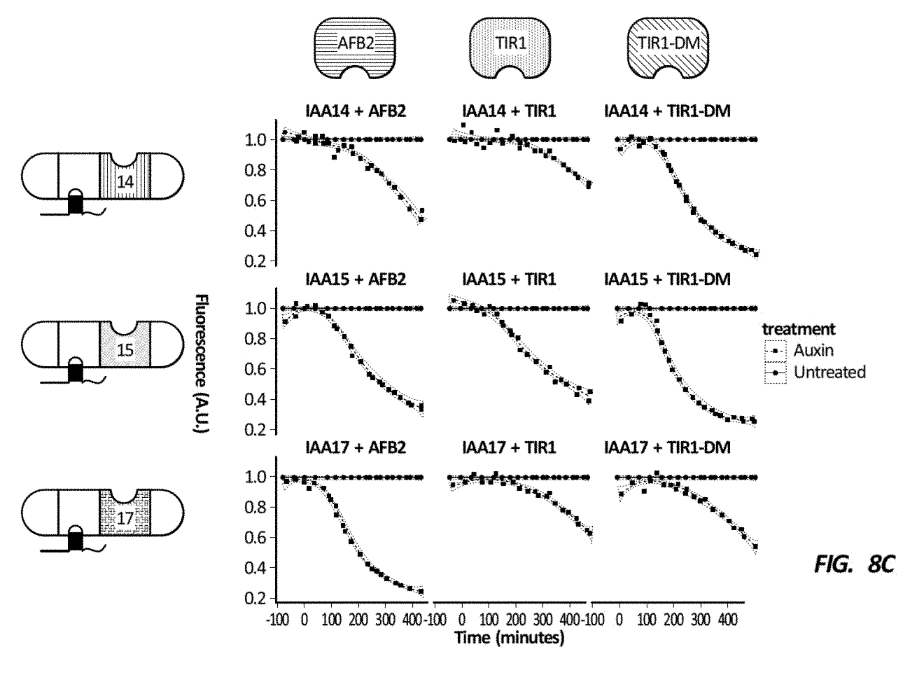

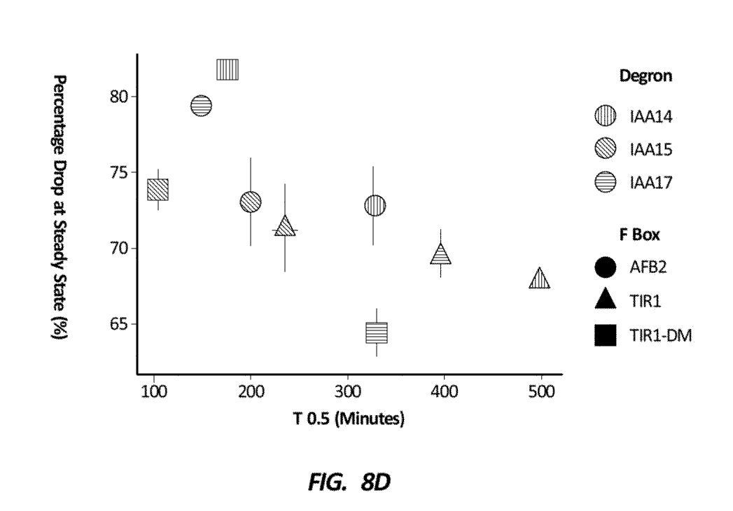

All pairwise combinations of ADCTFs and F-box proteins were tested for their temporal response and dose response to auxin (see FIG. 2A). Temporal responses, all performed with 30 μM auxin induction, exhibited a range of degradation kinetics that depended on both the choice of ADCTF degron and the F-box protein (see FIG. 2B). The kinetics, characterized by the time to 50% degradation, can be coarsely tuned by the choice of F-box protein used, with tir1-D/M being the fastest overall, followed by AFB2 and TIR1. Within this coarse tuning, the choice of degron allowed for smaller changes in kinetics. The ADCTF with the degron from IAA15 (ADC15) seemed to have the overall fastest kinetics. The only exception to this trend was the interaction between AFB2 and ADC17, which had the fastest degradation rate. All the ADCTFs had approximately the same percentage change from maximal activation upon auxin induction at steady state. Thus, tuning kinetics by swapping F-box proteins or degrons had a minimal effect on the steady state response to auxin. Most variants dropped to approximately 75% of maximal activation at steady state with a few between 10% higher or lower than the mean (see FIG. 8D). The ADCTFs exhibited varied sensitivity to auxin that depended on the combination of the degron on the ADCTF and the F-box protein. Swapping F-box proteins allowed for more coarse grain tuning of sensitivity range with TIR1 conferring the broadest sensitivity range overall and tir1-D/M conferring the narrowest (see FIG. 2C).

Swapping degrons allows smaller changes, as was observed within the AFB2 variants wherein there is a progressively narrower sensitivity range from ADC14 to ADC17. The dynamics and steady state behavior of the ADCTFs in response to auxin correspond to the behavior of previously characterized IAA proteins, from which the degrons were taken, in yeast (see Zhang, N.-N., et al. (2006) Mol. Biol. Cell 17, 3409-3422). The only exception being the degron 17 variant, which had much slower degradation kinetics in the ADCTF context in a tir1-D/M background. This result may suggest that the auxin responsive behavior can be predictably tuned by swapping degron and F-box protein variants.

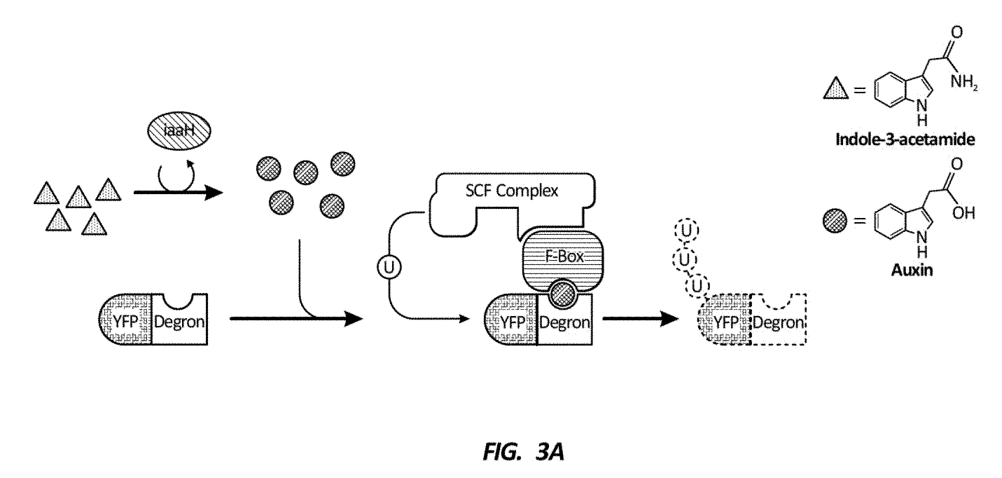

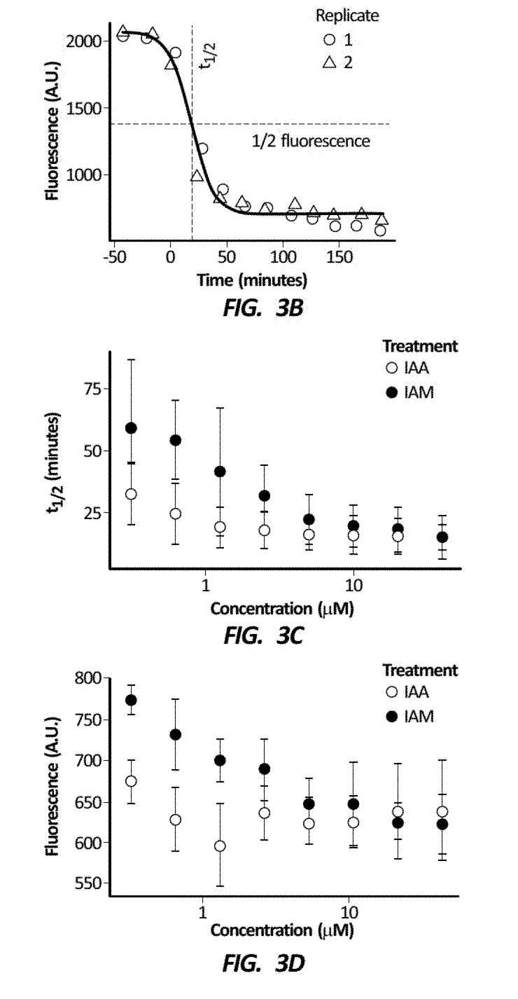

Yeast can produce tunable levels of auxin via expression of Iaah from Agrobacterium tumefaciens. To generate an auxin producing strain, half of the IAM pathway from Agrobacterium tumefaciens was integrated into yeast (see Zhao, Y. (2010) Annu. Rev. Plant Biol. 61, 49-64). The IAM pathway is a two-step enzymatic process that converts tryptophan to IAM and then into auxin. The first step is via tryptophan-2-monooxygenase (iaaM, not examined here). The second step is catalyzed by indole-3-acetamide hydrolase (iaaH). To test whether yeast could produce auxin from IAM using only the second enzyme, the iaaH gene from the Agrobacterium tumefaciens Ti plasmid (see P\check{a}curar, D. I., et al. (2011) Physiol. Mol. Plant Pathol. 76, 76-81) was integrated into an auxin reporting yeast strain (see FIG. 3A) containing a IAA-YFP fusion protein. After adding IAM, reporter degradation rates were measured via time-lapse cytometry (see FIG. 3B). Upon the addition of IAM, sender strains produced an auxin response comparable to that of native auxin (see FIG. 3C). In addition, for a given concentration of IAM, the steady state fluorescence values converge to those of auxin (see FIG. 3D). There was no significant delay between the addition of IAM and the production of auxin, so the transport and production of auxin from IAM can be assumed to be faster than the reporter's dynamics.

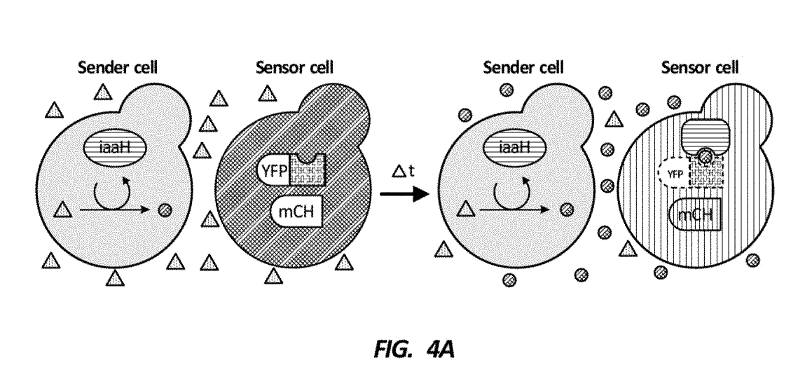

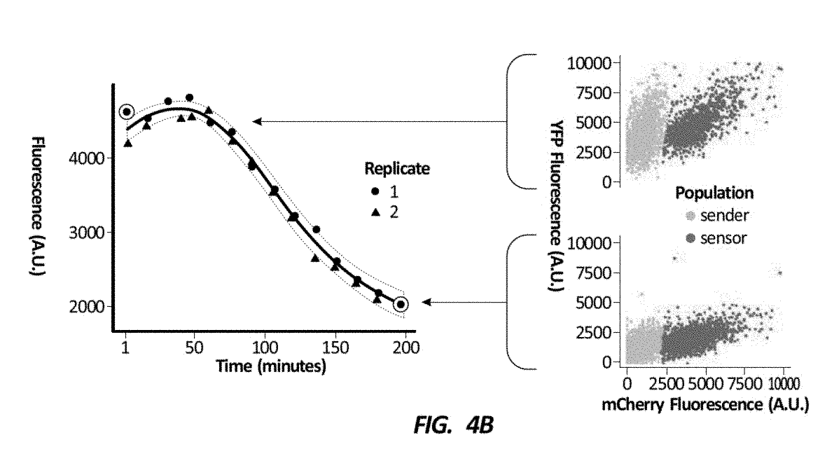

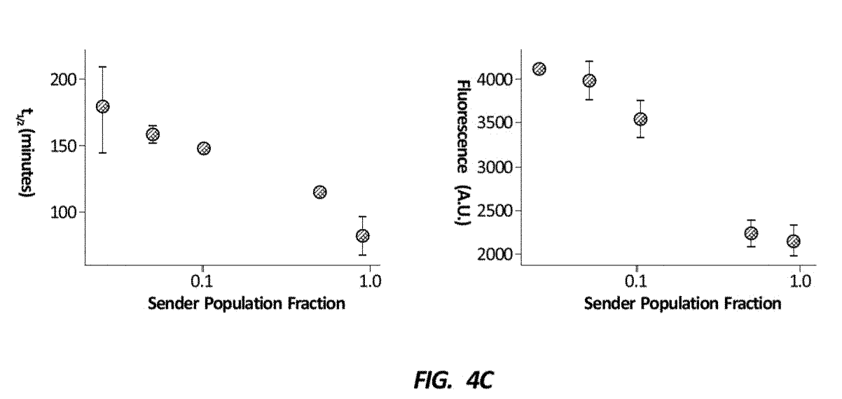

Intercellular auxin production was then investigated by coculturing the sender strain with an auxin sensor strain that could be distinguished via its mCherry signal (see FIG. 4A). Rather than a dose response of IAM, increasing fractions of sender cells were cocultured with sensor strains in a constant amount of the auxin precursor (300 μM) to test the dependence of auxin production on sender cell concentration (see FIG. 4B). Greater concentrations of sender cells produced a greater auxin response in sensor cells. Both the kinetic and steady state behavior suggest there is a lower concentration of auxin in the media than within sender cells (see FIG. 4C).

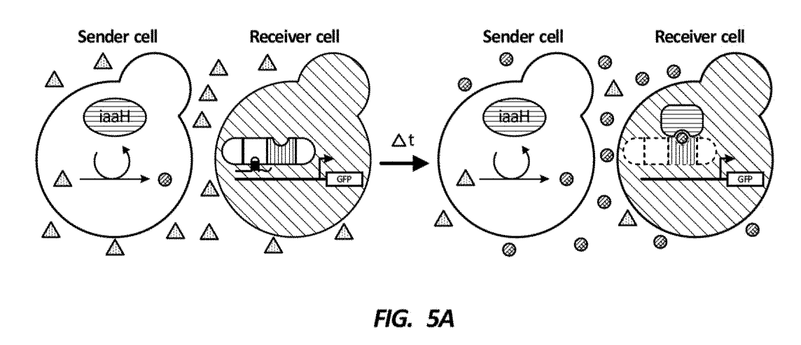

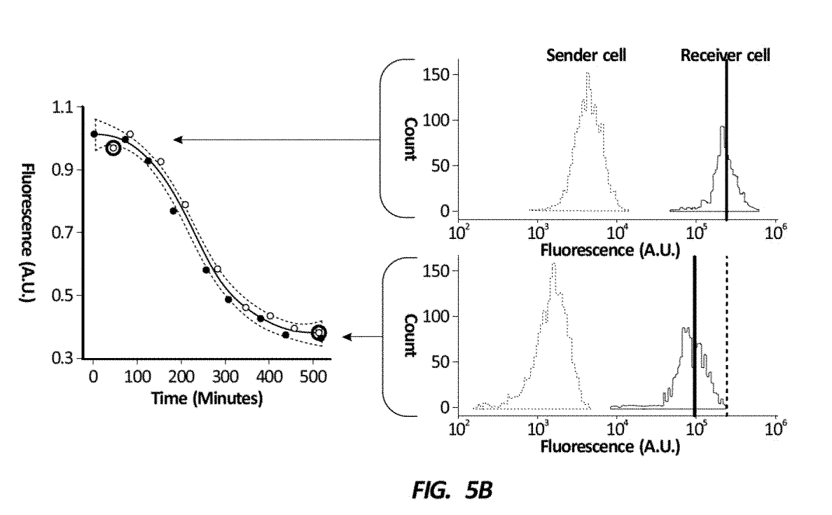

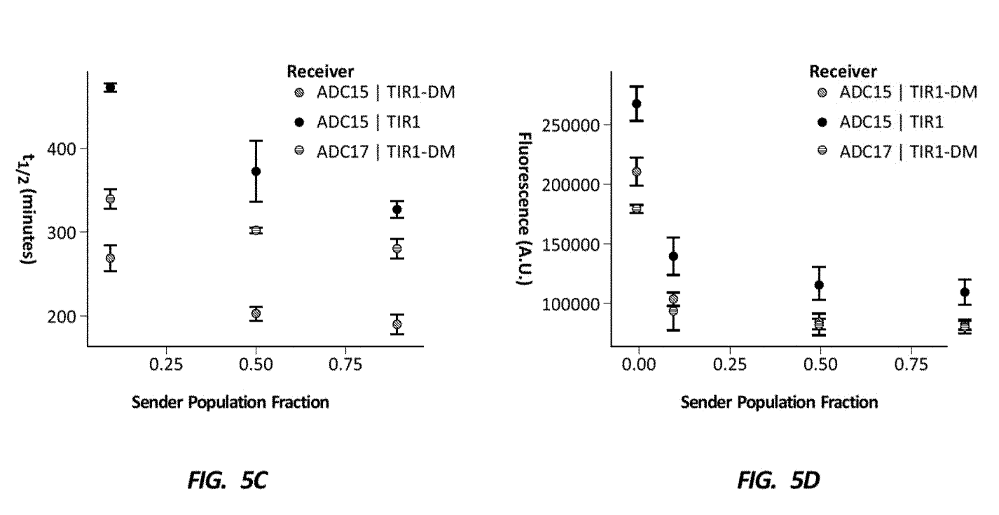

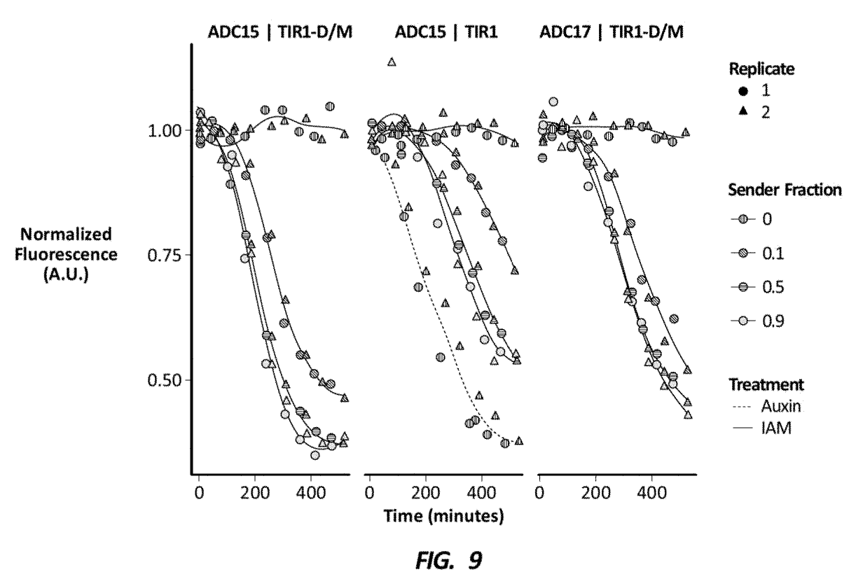

Sender cells can produce a tunable auxin response in receiver cells. Sender and receiver cells were cocultured in different ratios to measure the effect of sender cell concentration on auxin signal production. Senders constitutively express iaaH and the receivers expressed an activating ADCTF and a gRNA targeting a minimal CYC1 promoter driving EGFP (see FIG. 5A). After adding a saturating amount of the IAM and growing the coculture overnight, a reduction in gene activation was observed in the receiver strain comparable to direct addition of auxin (see FIG. 5B). Three different receiver strains with a range of responses to auxin were tested with the sender strain. All the receiver strains produced an auxin response and behaviors were consistent to those observed via the direct addition of auxin, suggesting that the sender module is compatible with any ADCTF-based receiver module (see FIG. 9). In addition, a 10% fraction of sender cells is sufficient for a significant change in fluorescence in receiver cells at steady state and a 50% fraction produces a nearly saturating signal (see FIGS. 5C and 5D).

The system provided herein is based on a signal transduction modality that is unique to plants and so is orthogonal to native yeast signal transduction pathways, as well as to mammalian cells (see Nishimura, K., et al. (2009) Nat. Methods 6, 917-22). The ADCTF library can allow the generation of a range of responses to the same auxin signal, and can in principle be connected to any gene of interest, or to another synthetic gene circuit. Additionally, auxin production levels can also be tuned by titrating in different amounts of IAM. It may also be possible to tune the diffusivity of auxin in yeast (see Zhao, Y. (2010) Annu. Rev. Plant Biol. 61, 49-64), or to harness the sequestration and turnover pathways of auxin found in plants. The approach provided herein of detecting small molecules via F-box mediated degradation of a transcription factor is potentially scalable as there are other plant hormones such as jasmonate that use a similar signaling pathway (see Turner, J. G., et al. (2002) Plant Cell 14 Suppl, S153-S164). Furthermore, feedback systems can be built through regulation of the iaaH gene via the ADCTFs. More generally, the system provided herein can form a basis platform for implementing distributed decision making, pattern formation, and other complex cell-to-cell communication based multicellular behaviors.

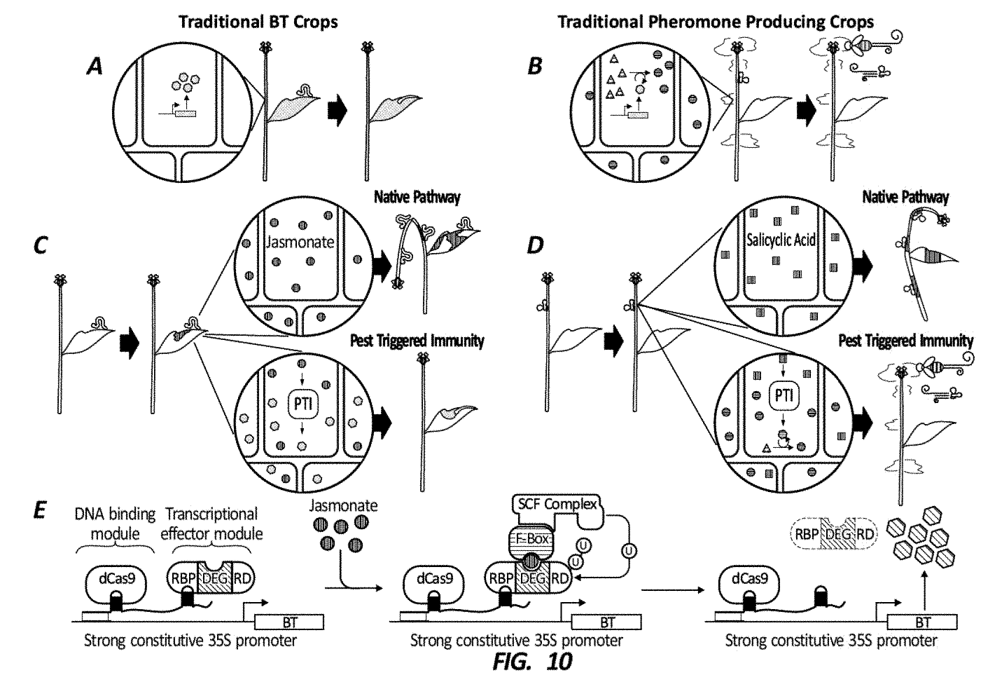

To address the drawbacks of constitutive expression strategies, the expression of insect resistance mechanisms can be linked to the endogenous hormonal cues associated with insect attack using synthetic phytohormone-based signal transduction machinery. By fusing hormone regulated degradation motifs to CRISPR-based transcription factors (CTFs) a platform for pest triggered immunity may be formed that can be ported from model systems to crops. CTFs have recently become a widely used technology in a variety of organisms, including A. thaliana (see Gilbert, L., et al. Cell 154, (2013); Farzadfard, F., et al. ACS Synthetic Biology 2, 604-613 (2013); Qi, L., et al. Cell 152, (2013); and Piatek, A., et al., “RNA-guided transcriptional regulation in planta via synthetic dCas9-based transcription factors,” Plant Biotechnology Journal (2014)). The inducible nature of such a system may increase yields as well as slow down the development of insecticide resistance in pests, resulting in safer and more productive crops (see FIG. 10, panels C and D).

The provided strategy for building phytohormone sensitive transcription factors utilizes modular domains for DNA binding, phytohormone response, and/or transcriptional regulation allowing independent tuning of each function. The dCas9 protein is used for DNA binding; accordingly, the transcription factor can be easily retargeted by swapping gRNAs. This can make the system flexible enough to be used to regulate a diverse array of genetically encoded insect defense mechanisms such as toxin production or volatile biosynthesis that have already been engineered into plant lines under constitutive promoters. Additionally, as the proposed mechanism relies on phytohormone signals and responsive elements that are widely conserved across plants as well as non-native proteins like dCas9 the system may be portable from a model system into relevant crop plants.

The synthetic signal transduction system may also interface with native hormonal cues in a multicellular organism. The system may also be applied to study other hormone regulated processes such as growth and development in both plants and mammalian systems, as well as to design novel therapies for hormone dysregulation pathologies such as diabetes that rely on fixing damaged pathways with synthetic components. The method of synthetic phytohormone responsive signal transduction may provide a framework for engineering hormone-based signal transduction in higher organisms. The method may also represent a novel approach to engineering pest resistance systems in plants and how plants interact with their environment.

A set of phytohormone responsive CRISPR-based transcription factors may be built. These transcription factors can utilize the dCas9 protein (see Gilbert, L., et al. Cell 154, (2013)) as a programmable DNA binding domain to recruit transcriptional effectors to regulate the expression of insect defense mechanisms. The transcription factors can be engineered to be hormone sensitive by fusing hormone triggered degradation domains (see Browse, J., Annual Review of Plant Biology 60, 183-205 (2009); Fu, Z Q, et al. “NPR3 and NPR4 are receptors for the immune signal salicylic acid in plants,” Nature (2012); and Joo, S., et al. The Plant Journal 54, 129-140 (2008)) to the transcriptional effectors (see FIG. 10, panel E).

The dCas9 protein in complex with a gRNA can act as an easily reprogrammable DNA binding domain. This gRNA can also act as a scaffold and recruit additional transcriptional regulation proteins via aptamer-protein interactions (see Zalatan, J G, et al., “Engineering Complex Synthetic Transcriptional Programs with CRISPR RNA Scaffolds,” Cell (2015)). To make these transcription factors phytohormone regulated, phytohormone regulated degradation domains (degrons) can be fused to the transcriptional regulators recruited by the scaffold. Jasmonate isoleucine (JA) responsive degrons from the JAZ protein family (see Browse, J., Annual Review of Plant Biology 60, 183-205 (2009)), as well as two recently characterized salicylic acid (SA) responsive degrons from the NPR1 and ACS6 proteins (see Fu, Z Q, et al., “NPR3 and NPR4 are receptors for the immune signal salicylic acid in plants,” Nature (2012) and Joo, S., et al. The Plant Journal 54, 129-140 (2008)) from A. thaliana may be utilized.

The JA degrons can function by forming a complex with an adapter F-box protein (COI1) that recruits the E3-ubiquitin ligase machinery in the presence of JA, leading to ubiquitination and subsequent degradation of the degron tagged protein (see Browse, J., Annual Review of Plant Biology 60, 183-205 (2009)). The SA degron from NPR1 can cause degradation when the SA concentration in the cells is either very low or high, with low turnover at intermediate concentrations, via two different F-box-like receptors, NPR4 and NPR3, respectively (see Fu, Z Q, et al., “NPR3 and NPR4 are receptors for the immune signal salicylic acid in plants,” Nature (2012)). The degron from ACS6 protein generally causes ACS6 to be turned over but is stabilized upon phosphorylation by the SA activated kinase SIPK (see Joo, S., et al. The Plant Journal 54, 129-140 (2008)).

Phytohormone degradable TFs can be used to link the flux of phytohormones produced within minutes of an insect attack on a plant to the expression of defense mechanisms. The relative concentration of these different hormones tends to be plant and pest specific, with different kinds of pests, such as phloem feeders like aphids versus leaf chewers like caterpillars, eliciting different hormonal profiles (see Vos, D. M., et al., “Signal signature and transcriptome changes of Arabidopsis during pathogen and insect attack,” MPMI Vol. 18, No. 9, 2005, pp. 923-937). However, certain trends are conserved across most plants. It has been shown that mechanical damage to tissues by pests causes a systemic JA response in the plant within minutes, starting from the point of attack (see Herde, M., et al., “Elicitation of jasmonate-mediated defense responses by mechanical wounding and insect herbivory,” 51-61 (2013) and Erb, M., et al., “Role of phytohormones in insect-specific plant reactions,” Trends in Plant Science 17, (2012)). The present synthetic signal transduction system can be utilized to link this pest induced jasmonate response to the production of a BT, Cry1Ab, which has been shown to be effective against caterpillars.

Certain pests, such as phloem feeders, do not cause as much mechanical damage and so do not elicit a strong JA response until a severe infestation occurs. However, other hormones such as SA have been shown to be upregulated in the presence of these pests (see Vos, D. M., et al., “Signal signature and transcriptome changes of Arabidopsis during pathogen and insect attack,” MPMI Vol. 18, No. 9, 2005, pp. 923-937 and Li, Q, et al., “Mi-1-mediated aphid resistance involves salicylic acid and mitogen-activated protein kinase signaling cascades,” MPMI Vol. 19, No. 6, 2006, pp. 655-664). Accordingly, it may be desirable to integrate different hormonal cues such as SA to more effectively deal with these pests. Phloem feeders are also not susceptible to BT so different defense mechanisms must be employed. An alternative strategy to BT-based toxins which is rapidly gaining popularity as a way to deal with pests is the idea of push-pull pest control systems (see Pickett, J., et al., “Push-pull farming systems,” Current Opinion in Biotechnology 26, (2014)). This idea revolves around the concept of engineering biosynthesis pathways for volatiles (see Schnee, C., et al., Proc. Natl. Acad. Sci. U.S.A 103, 1129-34 (2006) and Kappers, I., et al., Science 309, 2070-2072 (2005)) and/or insect pheromones (see Beale, M., et al. Proceedings of the National Academy of Sciences 103, 10509-10513 (2006) and Ding, B.-J., et al., “A plant factory for moth pheromone production,” Nature Communications 5,-(2014)) into plants to scatter feeding herbivores and recruit the natural predators of herbivores to the plants being attacked. Beale, et al. demonstrated that biosynthesis of the aphid alarm pheromone in A. thaliana could both scatter the herbivorous aphids as well as recruit their natural predators (see Beale, M., et al., Proceedings of the National Academy of Sciences 103, 10509-10513 (2006)).

In certain embodiments, the concept of a pest triggered immunity system may become especially important for the application of the present methods to large scale agriculture, as pests have been shown to adapt and become insensitive to pheromone signals to which they are continuously exposed (see Dolzer, J, et al., “Adaptation in pheromone-sensitive trichoid sensilla of the hawkmoth Manduca sexta,” Journal of Experimental Biology (2003)). The system provided herein could be used to make these volatile or pheromone biosynthesis pathways pest-inducible by regulating the expression of enzymes in the biosynthesis pathway, potentially slowing down or eliminating this adaptation effect. The synthetic nature of the present system can insulate it against cross regulation by other plant pathways, due to the absence of multiple regulation motifs that are typically present on native transcription factors.

Easy retargeting of the CTFs to regulate arbitrary promoters and the ability of phytohormone degrons to perceive insect associated JA and SA fluxes that are largely conserved among different plants will make the proposed system easy to port from model systems to relevant crop plants. While the provided transcription factor may be constitutively expressed, it may be expressed at a much lower concentration than BT to be effective. Thus, a yield benefit may be expected. By spatially and temporally restricting the expression of the defense genes to the plant being attacked by pests, diversion of resources may be minimized and yield may be maximized. Environmental contamination may also be reduced with these insecticidal proteins and the development of resistance may be slowed. It has been shown that one of the best ways to slow down the development of resistance to insecticides is to limit temporal exposure of pests to the insecticide (see Bates, S., et al. Nature Biotechnology 23, 57-62 (2005)). Thus, the present system may represent a major step forward in agricultural pest management as it can slow the development of resistant pests as well as improve the yield of crops thereby improving food security.

A first aspect of the disclosure relates to synthetic signal transduction systems or HDCTFs. The synthetic signal transduction system can include a dCas9, a nuclear localization signal, a phytohormone degron, and/or a transcriptional regulation domain.

In some embodiments, the synthetic signal transduction system can be a fusion protein. For example, the dCas9, the nuclear localization signal, the phytohormone degron, and/or the transcriptional regulation domain can be bound, coupled, or fused to each other. The synthetic signal transduction system can form or be configured to form a complex with a gRNA. In certain embodiments, the phytohormone degron may be an auxin-sensitive degron, a gibberellin-sensitive degron, a jasmonate-sensitive degron, a strigolactone-sensitive degron, a karrikin-sensitive degron, an ethylene sensitive degron, a salicylic acid-sensitive degron, or any other suitable phytohormone degron.

In various embodiments, the dCas9 may be coupled to the nuclear localization signal and the phytohormone degron, and the phytohormone degron may be further coupled to the transcriptional regulation domain. In various other embodiments, the phytohormone degron may be coupled to the nuclear localization signal and the dCas9, and the dCas9 may be further coupled to the transcriptional regulation domain. In various other embodiments, the dCas9 may be coupled to the nuclear localization signal and the transcriptional regulation domain, and the transcriptional regulation domain may be further coupled to the phytohormone degron. Other arrangements or orders of the dCas9, nuclear localization signal, phytohormone degron, and/or the transcriptional regulation domain are also within the scope of this disclosure.

Another aspect of the disclosure relates to methods of generating a non-naturally occurring plant. The method may include coupling a dCas9, a nuclear localization signal, a phytohormone degron, and/or a transcriptional regulation domain to form an HDCTF and expressing the HDCTF in a plant.

In some embodiments, the method of generating a non-naturally occurring plant may also include coexpressing a gRNA with the HDCTF in the plant. The gRNA may target or be configured to target a promoter of a predetermined gene such that the HDCTF regulates expression of the predetermined gene. The method of generating a non-naturally occurring plant may also include coexpressing an F-box protein with the HDCTF in the plant. In some embodiments, the HDCTF may be a fusion protein. The HDCTF may form or be configured to form a complex with the gRNA. As discussed above, the phytohormone degron may be selected from at least one of an auxin-sensitive degron, a gibberellin-sensitive degron, a jasmonate-sensitive degron, a strigolactone-sensitive degron, a karrikin-sensitive degron, an ethylene sensitive degron, a salicylic acid-sensitive degron, or another suitable phytohormone degron.

In certain embodiments, the phytohormone degron may be sensitive to a given hormone. For example, in the presence of the given hormone, the HDCTF may be degraded or configured to be degraded. The HDCTF may be degraded or configured to be degraded via ubiquitin-mediated proteosomal degradation. The HDCTF may be degraded or configured to be degraded via other suitable methods of degradation.

In various embodiments, the phytohormone degron may be sensitive to a given hormone, wherein a route of exposure of the plant to the given hormone is selected from at least one of an exogenous hormone treatment, an endogenous hormone flux, or another suitable route of exposure.

The plant may be selected from one of a monocotyledonous plant, a dicotyledonous plant, or another suitable plant. For example, the plant may be selected from one of, but not limited to, rice, maize, wheat, rye, barley, millet, sorghum, peanut, cassava, banana, orange, mandarin, lemon, grapefruit, pomelo, potato, tomato, pepper, eggplant, cabbage, radish, cauliflower, rape, alfalfa, bean, pea, pumpkin, cucumber, melon, apple, quince, cherry, plum, apricot, peach, and cotton. In some embodiments, the plant may be Arabidopsis thaliana.

The HDCTF may alter or be configured to alter at least one of flower, fruit, leaf, root, seed, and/or shoot development in the plant. The HDCTF may also alter or be configured to alter another aspect of development in the plant. In some embodiments, the HDCTF may regulate or be configured to regulate expression of an insect resistance mechanism in the plant, for example, in response to an insect attack on the plant.

Another aspect of the disclosure relates to non-naturally occurring plants. The plant may include an HDCTF fusion protein comprising a dCas9, a nuclear localization signal, a phytohormone degron, and/or a transcriptional regulation domain. Furthermore, the HDCTF fusion protein may be expressed or configured to be expressed in the non-naturally occurring plant.

In certain embodiments, the non-naturally occurring plant may further include a gRNA that targets or that is configured to target a promoter of a predetermined gene. The gRNA may be coexpressed or configured to be coexpressed with the HDCTF fusion protein in the non-naturally occurring plant such that the HDCTF fusion protein can regulate expression of the predetermined gene. Furthermore, an F-box protein may be coexpressed or configured to be coexpressed with the HDCTF fusion protein in the non-naturally occurring plant.

The phytohormone degron may be sensitive to a given hormone. For example, in the presence of the given hormone the HDCTF fusion protein may be degraded or configured to be degraded. The HDCTF fusion protein may be degraded or configured to be degraded via ubiquitin-mediated proteosomal degradation. Other suitable methods of degradation of the HDCTF fusion protein are also within the scope of this disclosure.

As will be understood by one of ordinary skill in the art, each embodiment disclosed herein can comprise, consist essentially of, or consist of its particular stated element, step, ingredient, or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components, and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Definitions and explanations used in the present disclosure are meant and intended to be controlling in any future construction unless clearly and unambiguously modified in the following examples or when application of the meaning renders any construction meaningless or essentially meaningless in cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

### EXAMPLES

The following examples are illustrative of disclosed methods and compositions. In light of this disclosure, those of skill in the art will recognize that variations of these examples and other examples of the disclosed methods and compositions would be possible without undue experimentation.

### Example 1—Strain Construction

Building off the work of Farzadfard, et al. (see (2013) ACS Synth. Biol. 2, 604-13), the reporter was a yeast-enhanced green fluorescent protein driven by a truncated CYC1 promoter. This reporter was integrated at the URA3 locus in the genome of the W303-1A ADE2 strain of Saccharomyces cerevisiae and this reporter strain was used as the parent for all ADCTF strains. All gRNA was driven by an ADH1 promoter driven construct that consists of a gRNA flanked on each side by a hammerhead and an HDV ribozyme, facilitating expression from an RNA polymerase II promoter. All the gRNA constructs were integrated at the HIS3 locus. AFB2, TIR1, and tir1-D/M were integrated, respectively, at the LEU2 locus, and were driven by the GPD promoter. The ADCTFs were constructed by fusing an SV40 nuclear localization tag, a VP64 activation domain, and an auxin degron to a dCas9 protein from Streptococcus pyogenes.

The auxin degron used for all characterization, unless otherwise mentioned, was a truncation of the degron from IAA17 from Arabidopsis that was characterized previously to have the fastest speed of degradation in the presence of AFB2 degradation machinery (see Havens, K. a, et al. (2012) Plant Physiol. 160, 135-42). The other degrons used were the domain two regions from IAA14 and IAA15. The ADCTF was driven by a beta-estradiol inducible version of the GAL1 promoter integrated at the TRP locus in the genome in all strains (see McIsaac, R. S., (2011) Mol. Biol. Cell 22, 4447-59). The iaaH gene was amplified via PCR from the Ti plasmid of Agrobacterium tumefaciens and cloned via the GATEWAY™ method into a single-integrating HIS3 plasmid behind the strong TDH3 promoter. The integrating plasmid cassette was produced via digestion of the plasmid by PmeI and integrated into an auxin reporter strain via a standard lithium acetate transformation method (see Gietz, R. D., et al. (2002) Methods Enzymol. 350, 87-96).

### Example 2—Cytometry

All cytometry measurements were acquired with an ACCURI™ C6 cytometer with attached CSAMPLER™ apparatus using 488 nm and 640 nm excitation lasers and a 533 nm (FL-1: YFP/GFP) emission filter. For experiments involving steady-state ADC behavior (see FIGS. 6A-7B) cultures were grown overnight in SC media with 1 nM beta-estradiol for ADC expression induction. The next morning they were diluted down 1:100 and then grown up for 5 hours and then induced with varying auxin concentrations from stock solutions. These cultures were then grown overnight and fluorescence was measured the next day. For the replicates, untreated cultures from the previous day's experiments were used as overnights to begin the next day's cultures.

For time course experiments, cultures were grown overnight in SC media with 1 nM beta-estradiol for ADC expression induction. The next morning two sets of parallel cultures were set up by diluting down the overnights 1:100. These were then grown up for 5 hours and then induced with 30 μM auxin and reads were taken every 30 minutes for approximately seven hours. A final steady state reading was taken the next day.

Cytometry data were analyzed using custom R scripts and the FLOWCORE™ package by first gating for the yeast diploid population and then generating mean fluorescence values for every point. These datasets were then collectively fit using the LOESS fitting function in R and metrics and error were derived from these fits. For the recovery experiments, the same protocol was used except the overnight cultures were grown up in SC with 1 nM beta-estradiol and 30 μM auxin.

### Example 3—Strains

All receiver strains are mated diploids (see Table 1 below). They were constructed by mating a Mat-a strain that has a minimal CYC1 promoter driven yeGFP and a gRNA flanked by two ribozymes driven by the ADH1 promoter, with a Mat alpha strain that has the ADCTF driven by a modified GAL promoter that has a binding site for the Z4 zinc finger, an F-box driven by an ACT1 promoter and the beta-estradiol inducible transcription factor Z4EV that drives the production of the ADCTF.

TABLE 1

| Strain | Integrations |
|---|---|
| ADC14 + TIR1 | URA3 - pCYC1:yeGFP |
| | HIS3 - pADH1:RGR-C3gRNA |
| | TRP1 - pGAL(Z4):dCas9-IAA14- |
| | VP64 LEU2 - pGPD:TIR1 |
| | HO - pACT1:Z4EV |
| ADC15 + TIR1 | URA3 - pCYC1:yeGFP |
| | HIS3 - pADH1:RGR-C3gRNA |
| | TRP1 - pGAL(Z4):dCas9-IAA15- |
| | VP64 LEU2 - pGPD:TIR1 |
| | HO - pACT1:Z4EV |
| ADC17 + TIR1 | URA3 - pCYC1:yeGFP |
| | HIS3 - pADH1:RGR-C3gRNA |
| | TRP1 - pGAL(Z4):dCas9-IAA17- |
| | VP64 LEU2 - pGPD:TIR1 |
| | HO - pACT1:Z4EV |
| ADC14 + AFB2 | URA3 - pCYC1:yeGFP |
| | HIS3 - pADH1:RGR-C3gRNA |
| | TRP1 - pGAL(Z4):dCas9- |
| | IAA14-VP64 LEU2 - |
| | pGPD:AFB2 |
| | HO - pACT1:Z4EV |
| ADC15 + AFB2 | URA3 - pCYC1:yeGFP |
| | HIS3 - pADH1:RGR-C3gRNA |
| | TRP1 - pGAL(Z4):dCas9- |
| | IAA15-VP64 LEU2 - |
| | pGPD:AFB2 |
| | HO - pACT1:Z4EV |
| ADC17 + AFB2 | URA3 - pCYC1:yeGFP |
| | HIS3 - pADH1:RGR-C3gRNA |
| | TRP1 - pGAL(Z4):dCas9- |
| | IAA17-VP64 LEU2 - |
| | pGPD:AFB2 |
| | HO - pACT1:Z4EV |
| ADC14 + TIR1-DM | URA3 - pCYC1:yeGFP |
| | HIS3 - pADH1:RGR-C3gRNA |
| | TRP1 - pGAL(Z4):dCas9- |
| | IAA14-VP64 LEU2 - |
| | pGPD:TIR1-DM |
| | HO - pACT1:Z4EV |
| ADC15 + TIR1-DM | URA3 - pCYC1:yeGFP |
| | HIS3 - pADH1:RGR-C3gRNA |
| | TRP1 - pGAL(Z4):dCas9- |
| | IAA15-VP64 LEU2 - |
| | pGPD:TIR1-DM |
| | HO - pACT1:Z4EV |
| ADC17 + TIR1-DM | URA3 - pCYC1:yeGFP |
| | HIS3 - pADH1:RGR-C3gRNA |
| | TRP1 - pGAL(Z4):dCas9- |
| | IAA17-VP64 LEU2 - |
| | pGPD:TIR1-DM |
| | HO - pACT1:Z4EV |
| Sender | HIS3 - pGPD:iaaH |
| | TRP1 - pGPD:EYFP- |
| | IAA17 LEU2 - |
| | pGPD:AFB2 |
| Sensor | HIS3 - pGPD:mCherry-Stop |
| | TRP1 - pGPD:YFP-IAA17 LEU2 - |
| | pGPD:TIR1 |

### Example 4—Plasmids

The following plasmids were used: 1) pMOD_U_pCYC1:yeGFP, 2) pMOD_H_pADH1:RGR-C3gRNA, 3) pMOD_T_TRP-pGAL(Z4):dCas9-IAA14-VP64, 4) pMOD_T_TRP-pGAL(Z4):dCas9-IAA15-VP64, 5) pMOD_T_TRP-pGAL(Z4):dCas9-IAA17-VP64, 6) pGP5G-TIR1, 7) pGP5G-AFB2, 8) pGP5G-TIR1-DM, 9) pMODKan-HO-pACT1-Z4EV, 10) pGP8G-iaaH, 11) pGP4GY-IAA17, and 12) pGP8G-mCherry-Stop.

### Example 5—Generation of a Phytohormone Regulated CRISPR-Based Transcription Factor Library

A library of phytohormone regulated CRISPR-based transcription factors may be built and their ability to regulate transcription in Arabidopsis thaliana upon phytohormone induction may be demonstrated. Transcription factor variants may be screened, which have different repression domains, phytohormone responsive degradation motifs, and number of transcriptional effectors, to find variants with optimal performance. This performance can be defined as minimum expression in the uninduced state, maximum fold change upon induction, and fast response to induction and can be quantified by fluorescence microscopy.

CTFs fused to JA and SA responsive degradation motifs can be utilized to implement synthetic signal transduction. The transcription factor may consist of a dCas9 protein that will recognize a gRNA scaffold. The gRNA scaffold can in turn recruit transcriptional effector domains fused to a JA or SA degron and an RNA binding protein (see FIG. 10, panel E). An ideal transcription factor may prevent any transcription of the defense mechanism in the absence of the insect herbivore and quickly and strongly turn on expression in its presence.

To optimize this system, the modular components that compose it may be varied to find a combination that has the minimum leak in the uninduced state, the maximum fold change upon induction, and fastest response to induction. Another parameter that tuning may be demonstrated for, but not necessarily optimized, is the sensitivity range of the transcription factor to SA or JA signals.

### Example 6—Variation of Repression Domains

Repression domains can be varied to identify the domain that gives the strongest repression with the fastest recovery. Base lines of Arabidopsis thaliana can be constructed with a dCas9 protein, a gRNA scaffold, and a GFP reporter expressed constitutively. Variants of the transcriptional effector module can be constructed with a gRNA-aptamer binding protein (see Zalatan, J G, et al., “Engineering Complex Synthetic Transcriptional Programs with CRISPR RNA Scaffolds,” Cell (2015)) and a phytohormone sensitive degron (phytodegron) from Jas9 fused to a variety of repression domains with GS linkers in between each domain. The Jas9 degron may be used as it has been demonstrated to cause rapid degradation of proteins that it is fused to in the presence of JA (see Larrieu, A, et al., “A fluorescent hormone biosensor reveals the dynamics of jasmonate signalling in plants,” Nature Communications (2015)).

The repression domains to be used can be a mixture of repression domains that have been previously characterized from plants (the SRDX domain, the LUG domain, and the TPL domain) (see Mahfouz, M M, et al., “Targeted transcriptional repression using a chimeric TALE-SRDX repressor protein,” Plant Molecular Biology (2012); Gonzalez, D, et al., “The transcription corepressor LEUNIG interacts with the histone deacetylase HDA19 and mediator components MED14 (SWP) and CDK8 (HEN3) to repress transcription,” Molecular and Cellular Biology (2007); and Szemenyei, H, et al., “TOPLESS mediates auxin-dependent transcriptional repression during Arabidopsis embryogenesis,” Science (2008)) as well as certain strong repression domains from mammalian systems (the KRAB domain and the MXI1 domain) (see Gilbert, L., et al., “CRISPR-Mediated Modular RNA-Guided Regulation of Transcription in Eukaryotes,” Cell 154, (2013)). A negative control without a repression domain can also be built.

Constructs expressing these modules on constitutive, non-tissue specific promoters can be integrated into the genome of previously described base lines via agrobacterium mediated transformation. To characterize leak from these variants, expression of the GFP reporter in the lines with the various repression domains can be compared to the negative control. The GFP expression can be assayed via fluorescence microscopy images of the leaf, flower, and stem tissues. To characterize the kinetics of recovery upon induction, two assays can be used, one where a range of JA is applied ectopically to leaf tissue and one where JA production in the leaf is induced by wounding as described by Herde, et al. (see “Elicitation of jasmonate-mediated defense responses by mechanical wounding and insect herbivory,” 51-61 (2013)). GFP levels in treated tissues can then be assayed via fluorescence microscopy and compared to untreated controls. Images can be taken at regular time intervals post JA induction to determine the kinetics.

Repression domains that give low leak and robust repression as compared to the control may be identified. A subset of these may have de-repression kinetics in the timescale of minutes to hours, such that the insect defense mechanism can retain effectiveness. Minimal repression in the negative control, which could be attributed to CRISPR interference (CRISPRi) or interference with the promoter's native activation machinery, may be identified.

Alternatively, tissue samples can be collected at regular intervals post induction and qPCR can be run to observe the GFP transcript level in the cell over time. Furthermore, a luciferase reporter may be used (instead of a fluorescent reporter) to avoid potential problems such as maturation time and background signal. If none of the domains show a release of regulation, weaker repression domains may be investigated.

### Example 7—Variation of the Phytodegron Motif

The phytodegron motif can be varied and the leak and kinetics of response post induction can be characterized for each variant. To characterize the phytohormone responsive degrons the transcriptional effector module variants can have a similar structure as described above with different phytodegron variants in place of Jas9. JA sensitive degrons from the 13 JAZ proteins identified so far can be used to engineer JA sensitivity (see Browse, J., Annual Review of Plant Biology 60, 183-205 (2009)). To engineer SA sensitivity, the entirety of the NPR1 protein and the C terminus of the ACS6 protein from A. thaliana can be used as SA responsive degrons. A control with no degron can also be built. To characterize the JA degrons, the same base plant lines as described above can be used. As the SA based degrons can cause degradation in the absence of SA, the transcriptional effector module can contain a VP64 activation domain instead of a repression domain. The base plant line to test them in can have a dCas9 and gRNA scaffold targeting a minimal, weakly expressing CMV promoter (see Bhullar, S., et al., Plant Biotechnology J 5, 696-708 (2007)) driving GFP. These modules can be integrated into the genome via agrobacterium mediated transformation and the resultant lines can be tested.

To assay the kinetics of response upon JA induction, all variants can be induced by wounding and a range of ectopically applied JA concentrations. To assay the kinetics of response upon SA induction, variants can be tested by a range of ectopically applied SA concentrations. Images of the test tissues can be taken before and at regular 15 minute intervals post induction to determine the kinetics of release of regulation. To assay the leak, each phytodegron variant can be compared to the no degron variant in the un-induced state in the leaf, stem, and flower tissue using fluorescence microscopy.

The phytodegrons may have a range of degradation rates and sensitivities to the phytohormone which can be apparent from the rate of recovery from repression of the reporter post phytohormone induction. Some of the degrons may have degradation rates in the timescale of minutes and physiologically relevant sensitivities as they are taken from phytohormone responsive proteins which demonstrate these properties. There may be range of leaks observed associated with the production of JA and SA transiently during development, but some degrons may be less sensitive to these transient hormone signals. If none of the phytodegron fusion constructs have response kinetics in the range that would be useful in the present system, the order of domains in the transcriptional effector module may be altered. Such a strategy has been shown to improve function.

### Example 8—Variation of the Number of Recruited Transcriptional Effector Modules

The number of transcriptional effector modules recruited to the scaffold can be varied to probe if increasing the number of recruited modules leads to higher fold change responses. Base plant lines that have the dCas9 and the optimal jasmonate induced transcriptional effector module integrated into the genome can be constructed. Plasmids encoding gRNA scaffolds that recruit zero, one, or two transcriptional effector modules, driven by constitutive non-tissue specific promoters can be transformed into the previously described base plant lines, which have all other components of the test circuit, via agrobacterium mediated transformation. The resulting plant lines can be induced by ectopic addition of a range of JA concentrations to leaf tissue and the fluorescence of the reporter can be assayed at regular intervals using fluorescence microscopy until a steady state is reached and compared to uninduced controls.

By increasing the number of recruited transcriptional effectors, the fold change upon induction may be increased significantly. The larger gRNA scaffold may be silenced in plants through their internal RNA degradation mechanisms. To work around this, the sequence of the gRNA may be varied by using different RNA aptamers, using silencing resistant structures, and potentially expressing RNAi-silencing suppressors from plant viruses (see Wang, M.-B., et al., Molecular Plant-Microbe Interactions 25, 1275-1285 (2012)).

### Example 9—Linking Herbivory to the Expression of Insect Defense Mechanisms

The optimal phytohormone regulated transcription factor variants can be used in Arabidopsis thaliana to link herbivory to the expression of insect defense mechanisms and quantify the benefit gained from pest induced immunity. The optimal insect responsive signal transduction system can be used to regulate both BT and aphid alarm pheromone production and the performance of these systems can be compared to constitutive and wild type controls. It can be aimed to verify that the optimized phytohormone responsive transcription factor behaves as expected in response to insect herbivory stimulated hormonal cues. It can also be aimed to assay the performance of pest triggered immunity plant lines versus controls with constitutive expression of defense mechanisms and without any defense mechanisms to demonstrate improved performance. Performance may be quantified according to three metrics: yield, measured by dry weight of the plants after herbivory; effectiveness, measured by the dry weight of pests collected post herbivory; as well as slower development of resistance among pests, quantified by the ratio of resistant to non-resistant pest populations post herbivory.

### Example 10—CRY1Ab (BT Toxin) Expression

A line of A. thaliana can be built in which CRY1Ab (BT toxin) expression is regulated by the optimal JA responsive transcription factor and its yield and resistance development potential can be tested via herbivory assays using the larvae of Pieris rapae and the diamond back moth. The plant line with the test circuit for the optimal JA responsive transcription factor can be transformed with a plasmid encoding the BT protein CRY1Ab codon optimized for Arabidopsis to minimize silencing (see Rocher, E., et al., Plant Physiology 117, 1445-1461 (1998)) (see FIG. 10, panel E). It can be driven by the same promoter as GFP so that the expression of the reporter can be an approximate readout for the expression of BT.

A control line can also be built with no gRNA expression making BT expression constitutive. These conditions along with a wild type negative control can be challenged in an herbivory assay as described by Herde, et al. (see “Elicitation of jasmonate-mediated defense responses by mechanical wounding and insect herbivory,” 51-61 (2013)). Each experiment can have plants that are treated with the insect herbivore and plants that are not treated, in order to recreate agricultural situations where only a subset of the crop plants are attacked by insects. The plants can be assayed post herbivory using fluorescence microscopy to determine whether the insect triggered hormonal flux is sufficient to cause the expression of GFP. The yield of these plants can be quantified by cleaning the plant tissue of any pests and measuring the dry weight of all plants in that trial post herbivory. The effectiveness can be measured during the same assay by collecting and measuring the dry weight of the pests post herbivory.

Cabbage butterfly (Pieris rapae) larvae can be used to challenge A. thaliana to characterize the yield and effectiveness of pest induced immunity versus constitutive immunity. This pest may be chosen as it is specialized to eat A. thaliana so its fitness will not be affected by native defense mechanisms (see Herde, M., et al., “Elicitation of jasmonate-mediated defense responses by mechanical wounding and insect herbivory,” 51-61 (2013)). To characterize the rate of development of resistance to the toxin, the plant lines can be challenged with an evenly mixed population of wild type and resistant diamond back moth larvae (see Cao, J, et al., “Transgenic broccoli with high levels of Bacillus thuringiensis Cry1C protein control diamondback moth larvae resistant to Cry1A or Cry1C,” Molecular Breeding (1999)) and a sample of the pests can be genotyped after several generations to determine the ratio of resistant to non-resistant pests.

A sufficiently large population of pests can be used to prevent immediate eradication of all non-resistant larvae. Comparable levels of effectiveness may be observed between the pathogen triggered immunity lines and the constitutive lines, however, a higher yield overall may be observed in the pathogen induced immunity lines as there is less diversion of resources.

There may be a less intense selection for the resistant allele in the pest induced immunity system than the constitutive line based on previous theoretical results (see Bates, S., et al., Nature Biotechnology 23, 57-62 (2005)). The effectiveness of the pest triggered immunity might be lower due to the delay between perception of the insects by the plants and the expression of the BT. Potentially, by building positive feedback into the system, the response may be sped up if necessary. If an immediate extinction of non-resistant pests is observed in the resistance development assay, a mixed population of test and wild type plants may be used in each trial.

### Example 11—Aphid Alarm Pheromone

A line of A. thaliana can be built in which the production of aphid alarm pheromone is regulated by the optimal SA responsive transcription factor and its yield can be tested via herbivory assays using the aphid Myzus persicae and their predator Diaeretiella rapae. The plant line with an optimal SA responsive transcription factor and a test circuit can be used as the base line. It can be transformed with a plasmid that encodes the aphid alarm pheromone synthesis gene, Eβf synthase (see Beale, M., et al., Proceedings of the National Academy of Sciences 103, 10509-10513 (2006)), driven by the same promoter as the reporter, giving a proxy for Eβf synthase expression. A constitutive control line with Eβf synthase expression driven by a strong constitutive promoter not targeted by the gRNA can also be built. These two lines and a wild type control can first be tested by challenging them with the phloem feeding pest (Myzus persicae) and then assaying for the expression of GFP in challenged tissues using fluorescence microscopy. They can then be tested in two formats to evaluate the effectiveness and yield, as defined above, of the pest induced immunity line compared to controls. Plants of each line will be exposed to the pest alone or both the pest and the predator (Diaeretiella rapae). The plants can be cleaned and their dry weight can be measured post herbivory to quantify yield from the lines under herbivory and herbivory with predation. The dry weight of the pests can be measured to quantify the effectiveness of each line.

To demonstrate the negative impact of adaptation in response to constitutive expression of the aphid alarm pheromone both pests and predators can be reared separately in the presence of each plant line over several generations. The pests and predators can then be released into a new growth chamber with fresh plants from the same lines both independently and together. The aversion of the pest to the plants and the attraction of the prey to the plants can be visually assayed and recorded when they are released separately. Additionally, when they are released together the dry weight of the pests and the plants can be assayed post herbivory to assay yield and effectiveness penalties associated with adaptation.

The yield from the pest triggered immunity plant strains may be significantly higher than the constitutive control as well as the wild type for at least two reasons. In the constitutive line, there may be a constant diversion of resources to the production of alarm pheromone which is metabolically taxing, whereas in the pest triggered immunity lines resources will only be diverted in the tissues under attack, and only during the period of the attack. Additionally, the continuous production of pheromone in the constitutive line may cause adaptation among the aphids and the predators, causing a decrease in its effectiveness as compared to the pest triggered immunity line.

A shift to the more controlled environment of an olfactometer to do the assays on adapted insects may be desirable to obtain data that has less experimental noise (see Knolhoff, L. M. et al., Annual Review of Entomology 59, 263-278 (2014).

### Example 12—Parallel Functionality of the Synthetic Phytohormone Signal Transduction Pathways

Parallel functionality of the synthetic phytohormone signal transduction pathways can be demonstrated through pest specific defense expression. Defense mechanisms to deal with both chewing and sucking herbivores can be engineered into one plant line, along with the insect responsive signal transduction systems regulating them. This line can then be assayed in the presence of each pest to validate that multiple insect responsive signal transduction systems can function in parallel to give pest specific responses. From this experiment it may be demonstrated whether pest specific hormone profiles can be linked to the expression of specific genes based on the changes in expression as compared against controls. It may also be assayed if the synthetic nature of the system allows for less crosstalk between different hormonal signal transduction pathways than natural systems.

### Example 13—Plant Line with Multiple Hormonal Regulated Defense Mechanisms

A plant line can be built that has both proposed hormonal regulated defense mechanisms and parallel and specific function can be demonstrated. To validate the pest specificity of present system a plant line can be built that has the optimal JA responsive transcription factor regulating expression of a GFP reporter and the optimal SA responsive transcription factor regulating expression of a CFP reporter. This may involve building constitutive promoters that have unique target sequences for the gRNAs in them so each gRNA-dCas9 complex is specific to one insect defense mechanism. This may be achieved by using the 35S promoter from the cauliflower mosaic virus and altering a 20 base pair sequence in the B2 region, which has been previously shown to be insensitive to mutation (see Szemenyei, H, et al., “TOPLESS mediates auxin-dependent transcriptional repression during Arabidopsis embryogenesis,” Science (2008)).

Negative control lines can also be built where the transcription factors do not have phytohormone degrons. These plant lines can then be challenged with different kinds of herbivory attack and the plants can be imaged post herbivory using florescence microscopy to assay the change in expression of the fluorescent protein. The plant lines can be exposed to chewing (Pieris rapae) herbivory and phloem sucking (Myzus persicae) herbivory, both separately and together. Attacked tissue can be imaged at 15 minute intervals post herbivory to characterize the response. An increase in GFP expression may be observed upon chewing herbivory and an increase in CYP expression may be observed upon phloem sucking herbivory due to the herbivory associated JA and SA fluxes, respectively.

### Example 14—Using Auxin Responsive Promoters to Engineer PIN1 Auxin Sensitivity

There are several mechanisms that play roles in plant developmental patterning, but the active transport of auxin from one tissue to another is central to this process. One of the most well studied and modelled developmental process in plants is phyllotactic patterning of flowers on the inflorescence meristem. In wildtype A. thaliana, the flowers are arranged in a spiral pattern with each one emerging at a 137.5° angle from the preceding flower, resulting in a spiral phyllotaxy. This is one of three predominant patterns observed in nature, the others being decussate where the flowers are at a 180° angle and whorled, where the flowers emerge in a ring at the same time. One of the major hypothesized molecular mechanisms that drives this patterning process is the feedback in auxin transport known as canalization. This process is largely driven by the auxin triggered expression of the auxin efflux protein PIN1. This feedback circuit results in polar auxin fluxes reinforcing themselves in plant tissues and results in the creation of auxin depletion zones around regions of auxin accumulation, such as initiating primordia. These depletion zones are also called inhibition zones as they prevent additional primordia from forming in them, thereby specifying a minimum spacing in between organs.

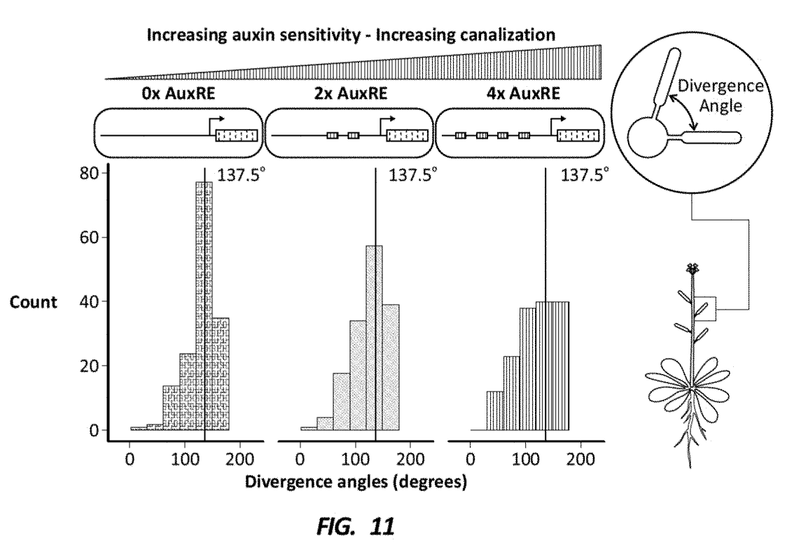

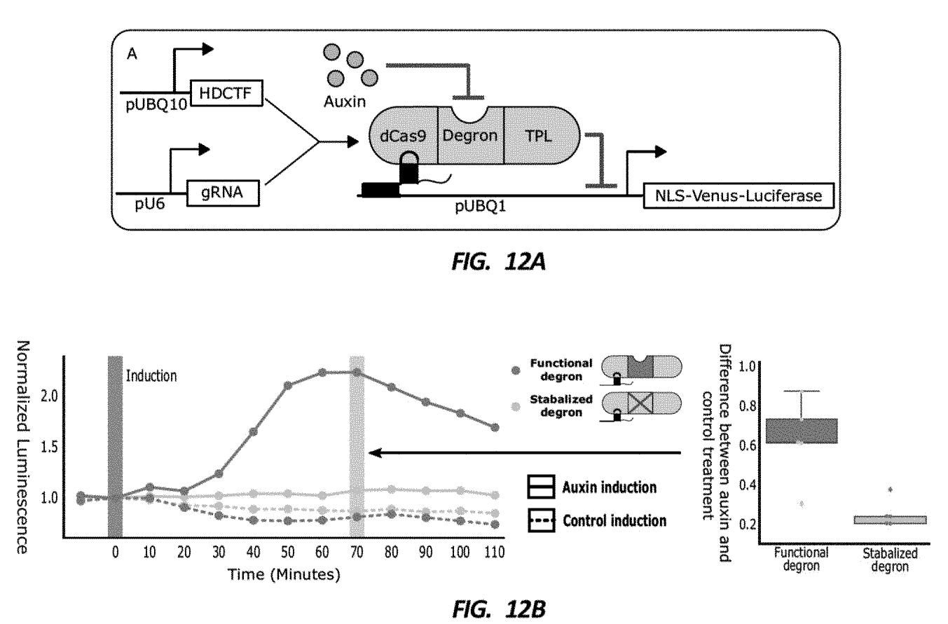

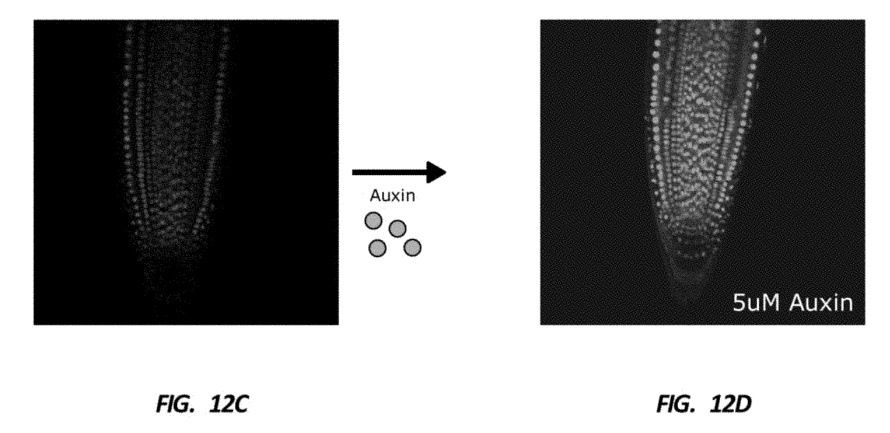

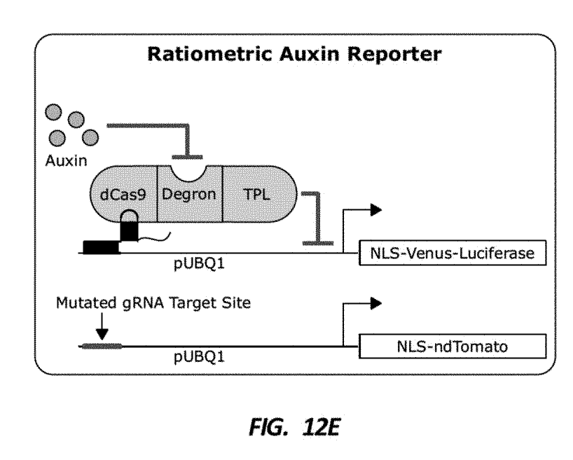

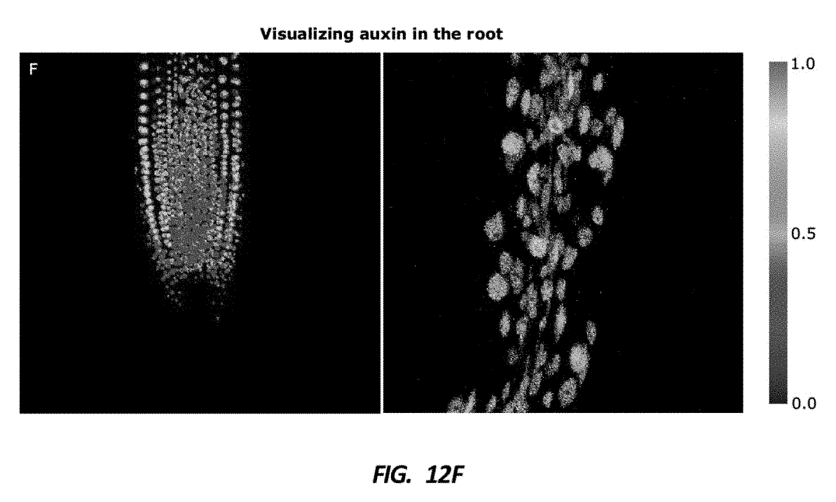

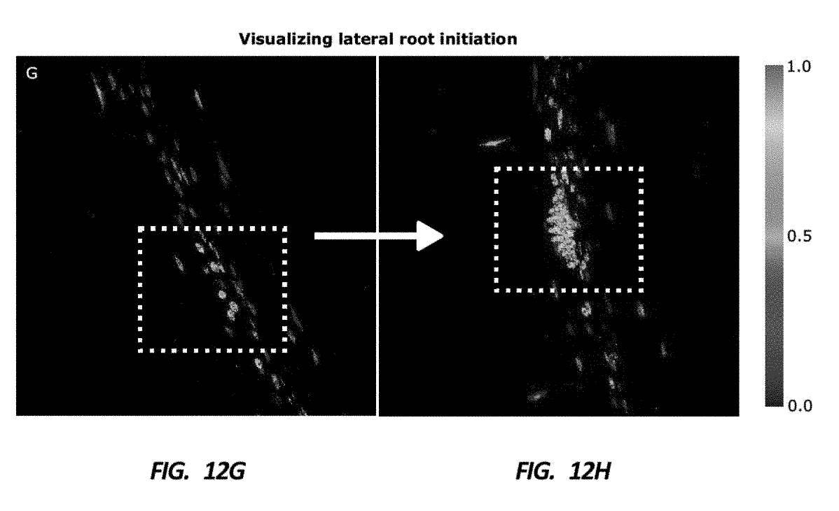

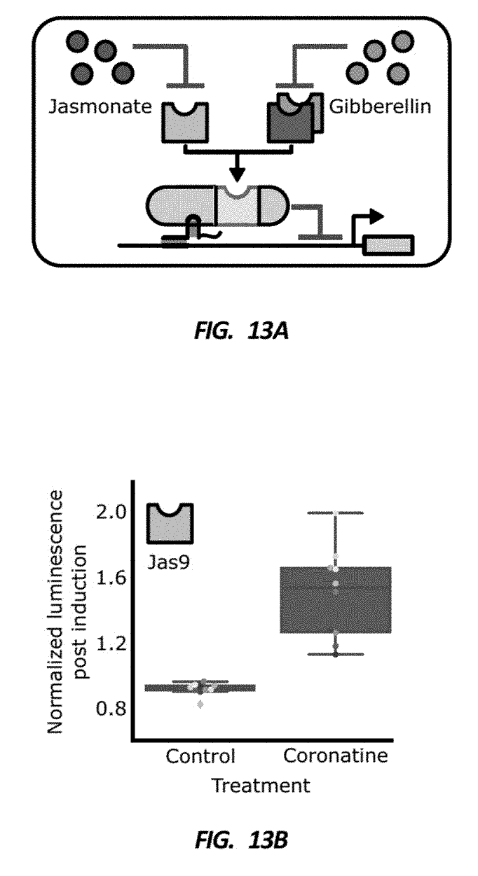

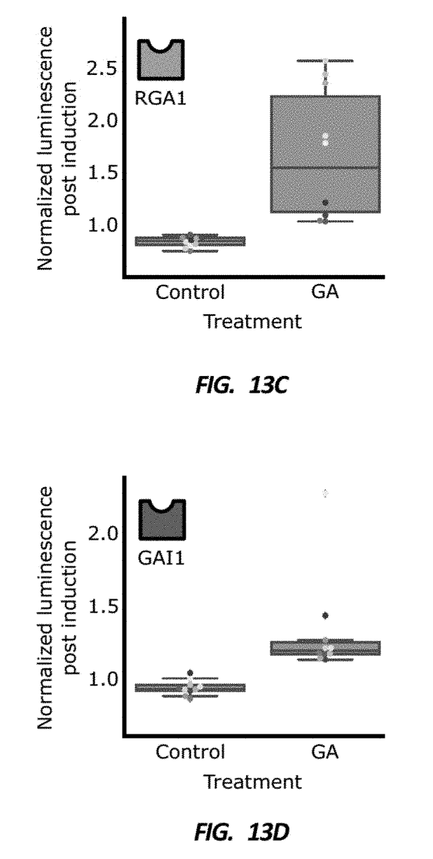

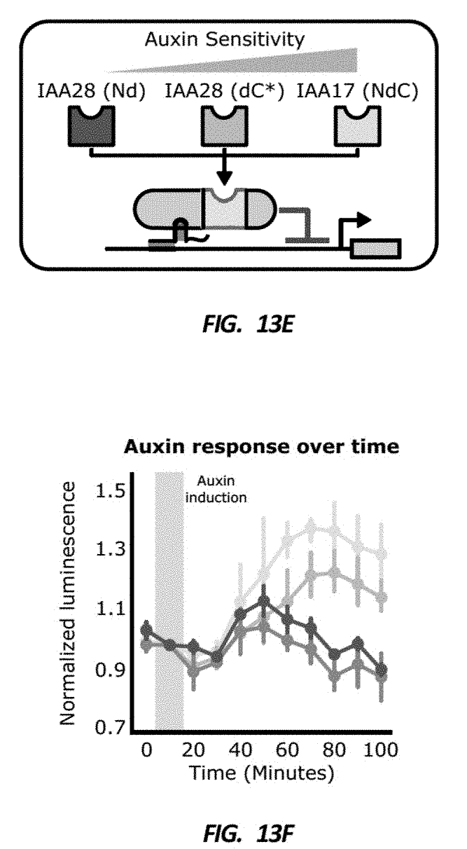

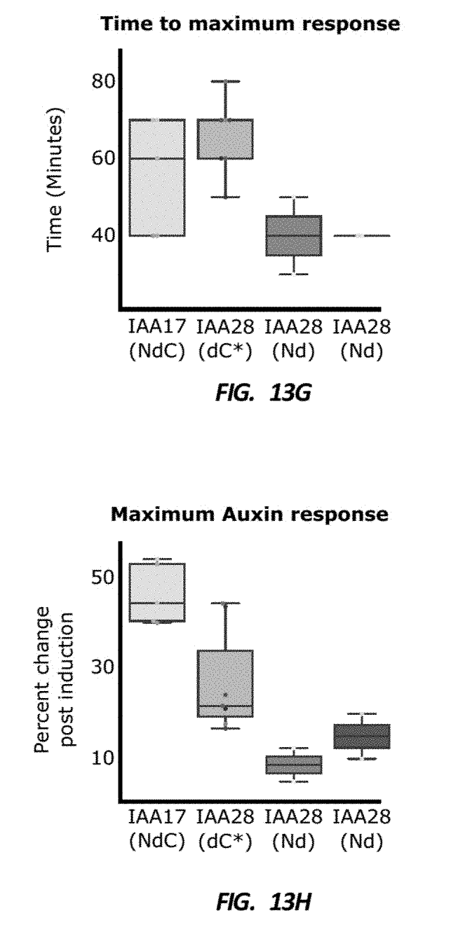

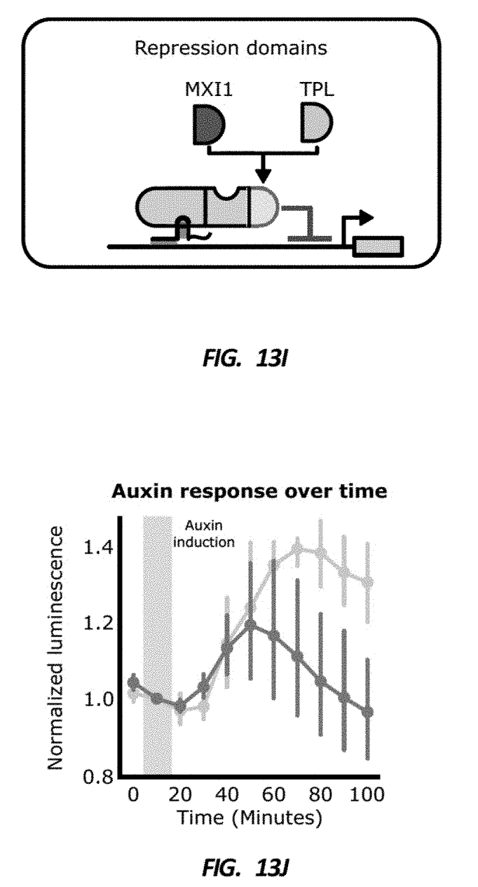

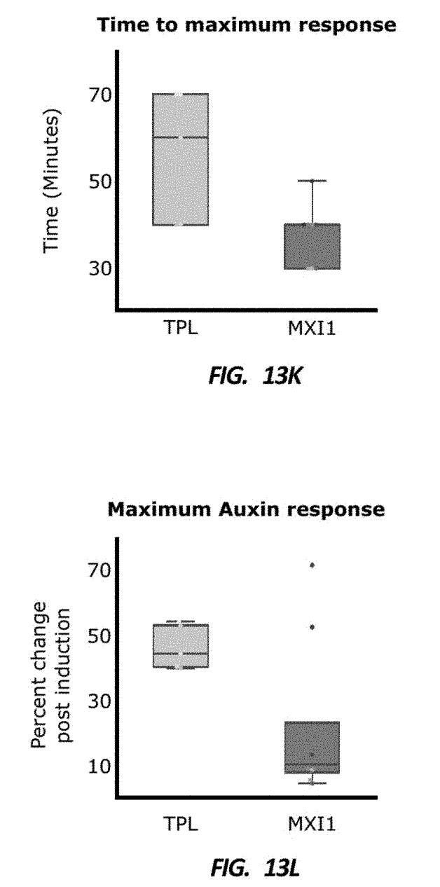

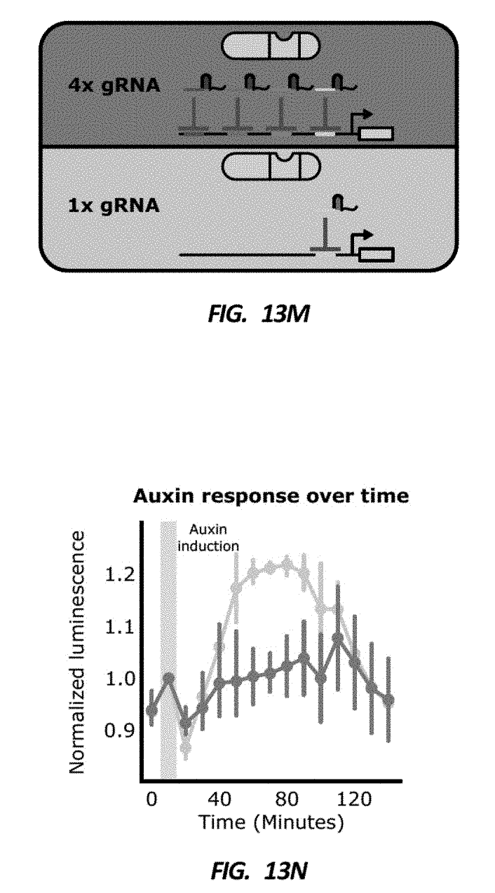

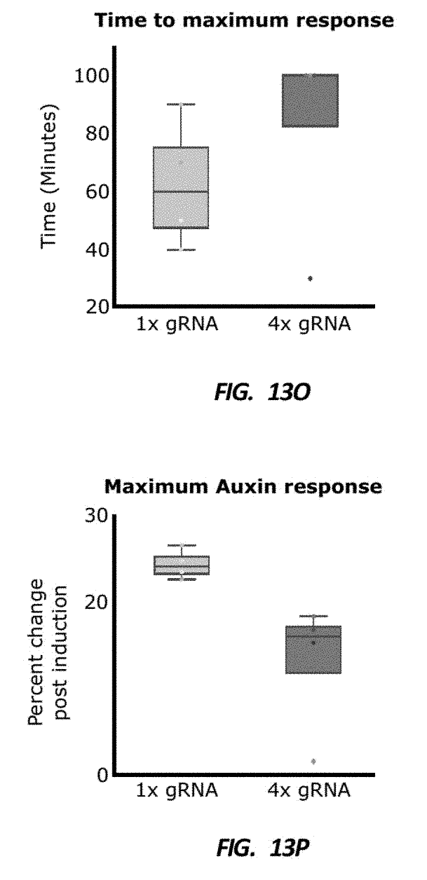

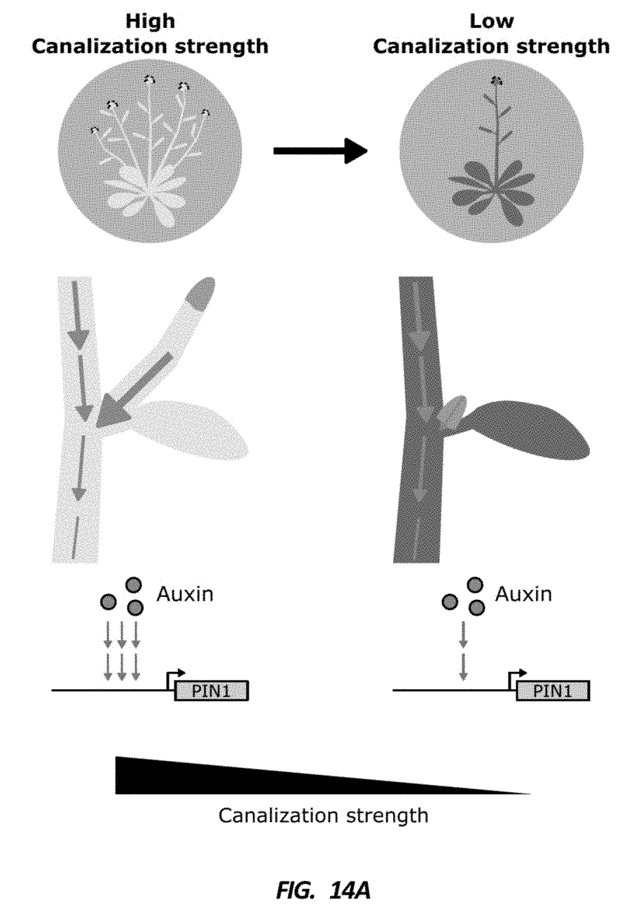

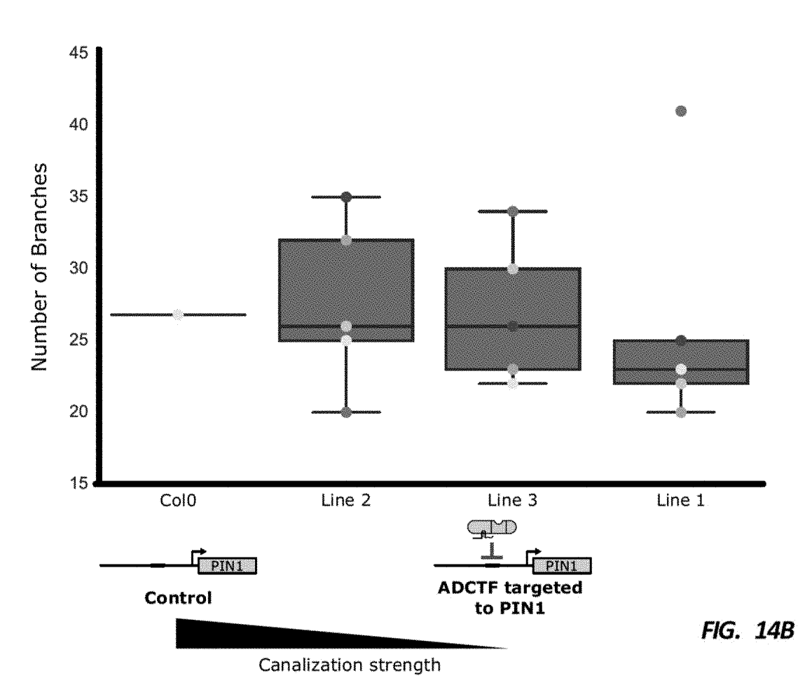

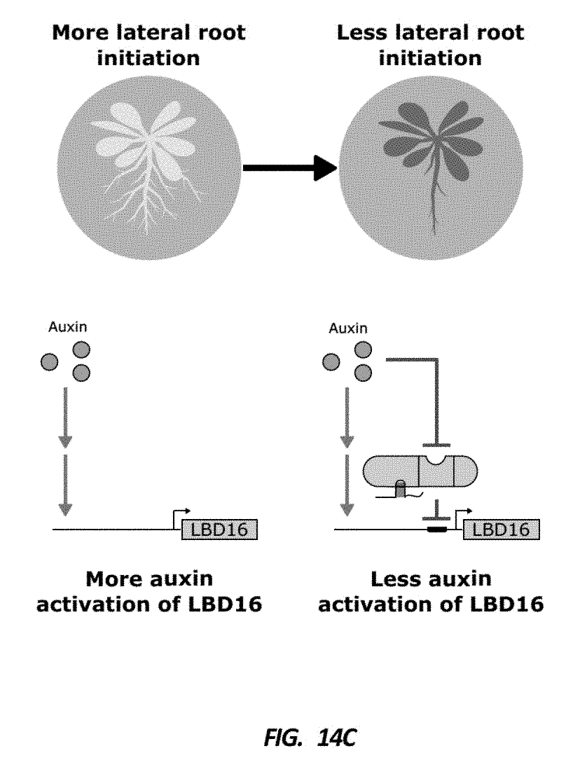

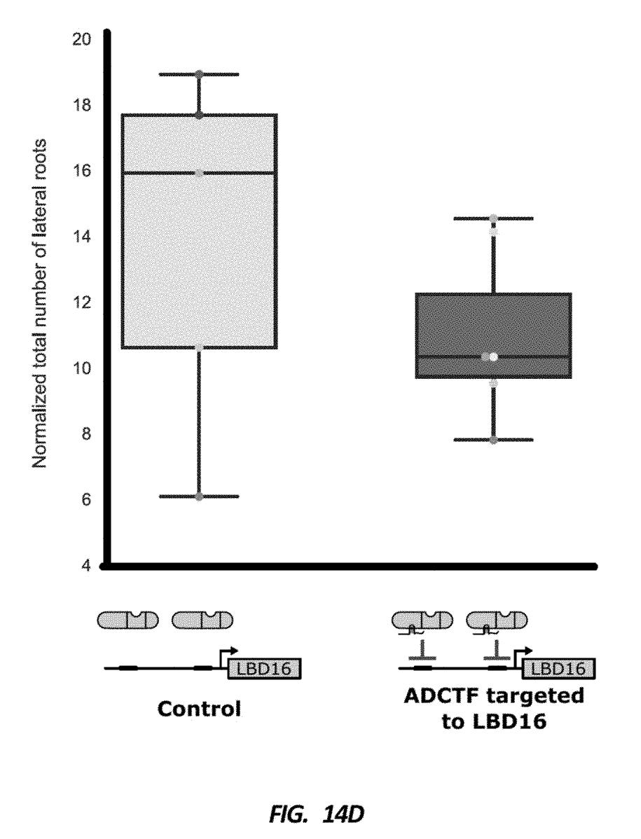

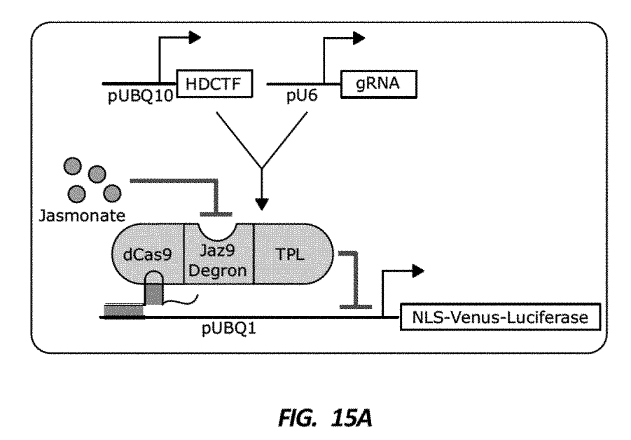

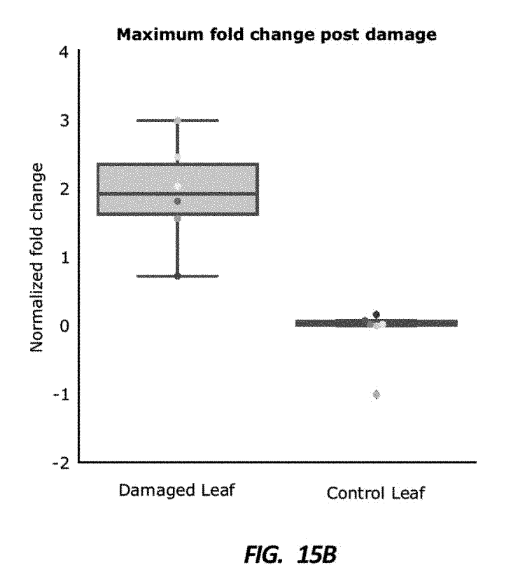

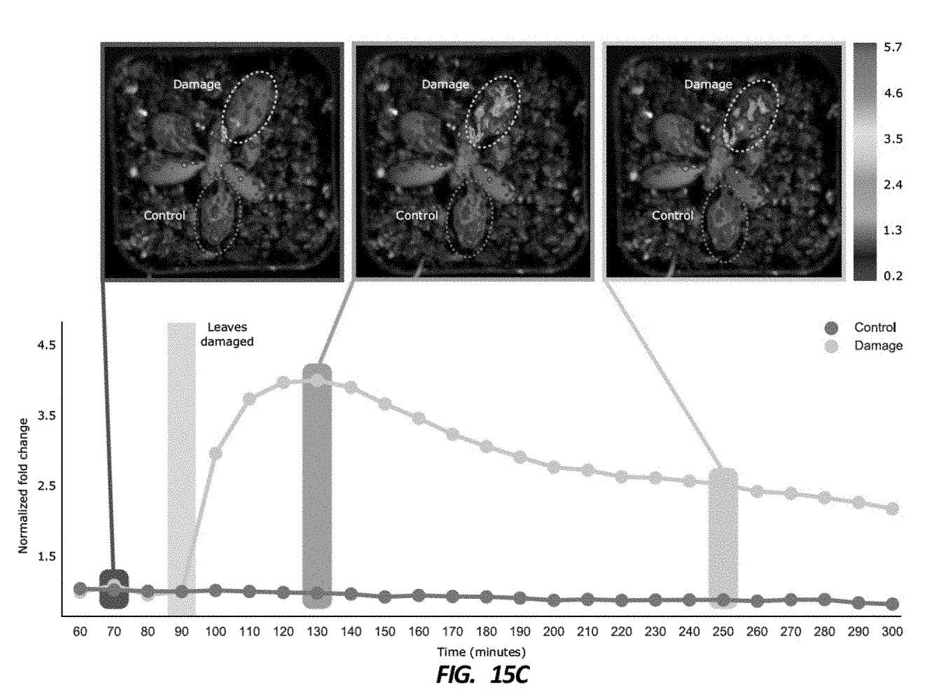

Models have shown that increasing the PIN1 driven canalization effect by increasing the auxin sensitivity of PIN1 expression is sufficient to change phyllotaxy, by increasing the noise and decreasing the average magnitude of the divergence angle between flowers. This would make PIN1 a target to engineer with the present ADCTF system. To test these model predictions, plant lines were built that had an extra copy of PIN1 driven by a set of synthetic promoters with different auxin sensitivities (see FIG. 11). The synthetic promoters had between zero and four auxin responsive cis regulatory elements (AuxREs) behind a 35S minimal promoter. Thus, plant lines were built in which the PIN1 driven canalization was progressively increased and it was observed that, as the models predict, there is a marginal shift to lower divergence angles and a dramatic increase in the noisiness of divergence angles, as evidenced by the wide spread in histograms of divergence angles.

Without being bound by any one specific theory, the overserved noisiness may be explained, in part, by the approach used here. The synthetic promoters retain none of the regulatory elements in the native PIN1 promoter, and so tissue specificity and temporal regulation are lost. These results validate PIN1 as a good target to regulate with the ADCTF system to control phyllotaxy.

Certain embodiments of this disclosure are described herein, including the best mode known to the inventors for carrying out the disclosure. Of course, variations on these described embodiments will become apparent to those of ordinary skill in the art upon reading the foregoing description. The applicants expect skilled artisans to employ such variations as appropriate, and the applicants intend for the various embodiments of the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents and printed publications throughout this specification. Each of the above-cited references and printed publications are individually incorporated herein by reference in their entirety.

It is to be understood that the embodiments of the present disclosure are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure.

It will be apparent to those having skill in the art that many changes may be made to the details of the above-described embodiments without departing from the underlying principles of the disclosure. The scope of the present invention should, therefore, be determined only by the following claims.

1. A synthetic signal transduction system comprising:
a nuclease null Cas9 protein (dCas9);
a nuclear localization signal;
a phytohormone degron; and
a transcriptional regulation domain.

2. The synthetic signal transduction system of claim 1, wherein the synthetic signal transduction system is a fusion protein.

3. (canceled)

4. The synthetic signal transduction system of claim 1, wherein the phytohormone degron is selected from at least one of an auxin-sensitive degron, a gibberellin-sensitive degron, a jasmonate-sensitive degron, a strigolactone-sensitive degron, a karrikin-sensitive degron, an ethylene sensitive degron, and a salicylic acid-sensitive degron.

5. The synthetic signal transduction system of claim 1, wherein the dCas9 is coupled to the nuclear localization signal and the phytohormone degron, and wherein the phytohormone degron is further coupled to the transcriptional regulation domain.

6. The synthetic signal transduction system of claim 1, wherein the phytohormone degron is coupled to the nuclear localization signal and the dCas9, and wherein the dCas9 is further coupled to the transcriptional regulation domain.

7. The synthetic signal transduction system of claim 1, wherein the dCas9 is coupled to the nuclear localization signal and the transcriptional regulation domain, and wherein the transcriptional regulation domain is further coupled to the phytohormone degron.

8. A method of generating a non-naturally occurring plant, the method comprising:
coupling a nuclease null Cas9 protein (dCas9), a nuclear localization signal, a phytohormone degron, and a transcriptional regulation domain to form a hormone degradable CRISPR-based transcription factor (HDCTF); and
expressing the HDCTF in a plant.

9. The method of claim 8, further comprising at least one of:
coexpressing a guide RNA (gRNA) with the HDCTF in the plant, wherein the gRNA is configured to target a promoter of a predetermined gene such that the HDCTF regulates expression of the predetermined gene, and
coexpressing an F-box protein with the HDCTF in the plant.

10. (canceled)

11. The method of claim 8, wherein the phytohormone degron is sensitive to a given hormone, and wherein in the presence of the given hormone the HDCTF is configured to be degraded.

12. (canceled)

13. The method of claim 8, wherein the phytohormone degron is sensitive to a given hormone, and wherein a route of exposure of the plant to the given hormone is selected from at least one of an exogenous hormone treatment and an endogenous hormone flux.

14-16. (canceled)

17. The method of claim 8, wherein the HDCTF is configured to alter at least one of flower, fruit, leaf, root, seed, and shoot development in the plant.

18. The method of claim 8, wherein the HDCTF is configured to regulate expression of an insect resistance mechanism in the plant in response to an insect attack on the plant.

19. The method of claim 8, wherein the HDCTF is a fusion protein.

20. (canceled)

21. The method of claim 8, wherein the phytohormone degron is selected from at least one of an auxin-sensitive degron, a gibberellin-sensitive degron, a jasmonate-sensitive degron, a strigolactone-sensitive degron, a karrikin-sensitive degron, an ethylene sensitive degron, and a salicylic acid-sensitive degron.

22. A non-naturally occurring plant comprising:
a hormone degradable CRISPR-based transcription factor (HDCTF) fusion protein comprising a nuclease null Cas9 protein (dCas9), a nuclear localization signal, a phytohormone degron, and a transcriptional regulation domain, wherein the HDCTF fusion protein is configured to be expressed in the non-naturally occurring plant.

23. The non-naturally occurring plant of claim 22, further comprising at least one of:
a gRNA configured to target a promoter of a predetermined gene, wherein the gRNA is configured to be coexpressed with the HDCTF fusion protein in the non-naturally occurring plant such that the HDCTF fusion protein regulates expression of the predetermined gene, and
an F-box protein configured to be coexpressed with the HDCTF fusion protein in the non-naturally occurring plant.

24. (canceled)

25. The non-naturally occurring plant of claim 22, wherein the phytohormone degron is sensitive to a given hormone, and wherein in the presence of the given hormone the HDCTF fusion protein is configured to be degraded.

26-29. (canceled)

30. The non-naturally occurring plant of claim 22, wherein the HDCTF fusion protein is configured to alter at least one of flower, fruit, leaf, root, seed, and shoot development in the non-naturally occurring plant.

31. The non-naturally occurring plant of claim 22, wherein the HDCTF fusion protein is configured to regulate expression of an insect resistance mechanism in the non-naturally occurring plant in response to an insect attack on the non-naturally occurring plant.

32. The non-naturally occurring plant of claim 22, wherein the phytohormone degron is selected from at least one of an auxin-sensitive degron, a gibberellin-sensitive degron, a jasmonate-sensitive degron, a strigolactone-sensitive degron, a karrikin-sensitive degron, an ethylene sensitive degron, and a salicylic acid-sensitive degron.
